(19) United States
(12) Patent Application Publication
(10) Pub. No.: US 2006/0148860 A1
(43) Pub. Date: Jul. 6, 2006

(54) Substituted p-phenyl carbamates

(75) Inventors: Per Vedso, Vaerlose (DK); Soren Ebdrup, Roskilde (DK); Poul Jacobsen, Slangerup (DK)

Correspondence Address: NOVO NORDISK, INC.;PATENT DEPARTMENT, 100 COLLEGE ROAD WEST, PRINCETON, NJ, 08540, US

(73) Assignee: Novo Nordisk A/S, Bagsvaerd (DK)

(21) Appl. No.: 11/299,982

(22) Filed: Dec. 12, 2005

Related U.S. Application Data
(63) Continuation of application No. PCT/DK04/00401, filed on Jun. 10, 2004.

(30) Foreign Application Priority Data
Jun. 12, 2003 (DK) PA 2003 00878

(51) Int. Cl. A61K 31/445; A61K 31/44; A61K 31/4166; C07D 401/02

(52) U.S. Cl. 514/346; 514/357; 514/398; 546/291; 548/324.5; 514/317

(57) ABSTRACT

Novel substituted p-phenyl carbamates, pharmaceutical compositions comprising them and use thereof in the treatment and/or prevention of diseases and disorders related to hormone sensitive lipase. More particularly, the compounds are useful for the treatment and/or prevention of diseases and disorders in which modulation of the activity of hormone sensitive lipase is beneficial.

### CROSS-REFERENCE TO RELATED APPLICATIONS

This application is a continuation of International Application No.: PCT/DK2004/000401, filed Jun. 10, 2004, which claims priority of Danish Patent Application No.: PA 2003 00878, filed Jun. 12, 2003, and U.S. Patent Application No.: 60/478,452, filed Jun. 13, 2003.

### FIELD OF THE INVENTION

The present invention relates to novel substituted p-phenyl carbamates, to pharmaceutical compositions comprising these compounds, to the use of these compounds as pharmaceutical compositions, and to methods of treatment employing these compounds and compositions. The present compounds show inhibition of hormone sensitive lipase. As a result, the compounds are useful for the treatment and/or prevention of diseases and disorders related to hormone sensitive lipase.

### BACKGROUND OF THE INVENTION

The overall energy homeostasis of a mammalian system requires a high degree of regulation to ensure the availability of the appropriate substrate at the appropriate time. Plasma glucose levels rise during the post-prandial state, to return to pre-prandial levels within 2-3 hours. During these 2-3 hours, insulin promotes glucose uptake by skeletal muscle and adipose tissue and decreases the release of free fatty acids (FFA) from adipocytes, to ensure that the two substrates do not compete with each other. When plasma glucose levels fall, an elevation in plasma FFA is necessary to switch from glucose to fat utilization by the various tissues.

In individuals with insulin resistance, FFA levels do not fall in response to insulin, as they do in normal individuals, preventing the normal utilization of glucose by skeletal muscle, adipose and liver. Furthermore, there is a negative correlation between insulin sensitivity and plasma FFA levels.

Hormone-sensitive lipase (HSL) is an enzyme, expressed in adipose tissue, macrophages, muscle, adrenal, testis and islets (Kraemer and Shen, J. Lipid Res. 2002, 43, 1585-1594). In the adipocytes HSL catalyses the conversion of triglycerides to glycerol and fatty acids. It is through the regulation of this enzyme that the levels of circulating FFA are modulated. Insulin leads to the inactivation of HSL with a subsequent fall in plasma FFA levels during the post-prandial state, followed by the activation of the enzyme when the insulin concentration falls and catecholamines rise during the post-absorptive period. The activation of HSL leads to an increase in plasma FFA, as they become the main source of energy during fasting.

The activation-inactivation of HSL is primarily mediated through the cAMP-protein kinase A and AMP-dependent kinase pathways. There are compounds like nicotinic acid and its derivatives, that decrease the activation of HSL via these pathways and cause a decrease in lipolysis that leads to a reduction in the FFA levels. These drugs have a beneficial effect in the utilization of glucose and in the normalization of the excess triglyceride synthesis seen in patients with elevated FFA. However, since these pathways are used by other processes in the body, these drugs have severe side effects.

Several publications disclose the preparation and use of HSL inhibitors (WO 01/87843, WO 01/17981, WO 01/66531, WO 01/83497, and WO 01/26664). However, the structures of these compounds are very different from that of the present compounds. Thus, none of the HSL inhibitors disclosed in these publications contain phenyl and carbamate substructures as in the compounds of the present invention.

We have found p-phenyl substituted carbamate compounds that specifically inhibit the lipolytic activity of HSL and which may be expected to decrease plasma FFA levels. These compounds can be used to treat disorders where a decreased level of plasma FFA is desired, such as insulin resistance, syndrome X, dyslipidemia, abnormalities of lipoprotein metabolism.

One aspect of the present invention is to provide compounds and pharmaceutical compositions that inhibit the lipolytic activity of HSL. A further aspect is to provide compounds which have good pharmaceutical properties such as solubility, bioavailability, specificity etc.

### DEFINITIONS

The following is a detailed definition of the terms used to describe the compounds of the invention.

The term “halogen” in the present context designates an atom selected from the group consisting of F, Cl, Br and I.

The tern “C₁₋₆alkyl” in the present context designates a saturated, branched or straight hydrocarbon group having from 1 to 6 carbon atoms. Representative examples include, but are not limited to, methyl, ethyl, n-propyl, isopropyl, butyl, isobutyl, sec-butyl, tert-butyl, n-pentyl, iso-pentyl, neopentyl, tert-pentyl, n-hexyl, isohexyl and the like.

The term “C₂₋₆-alkyl” in the present context designates a saturated, branched or straight hydrocarbon group having from 2 to 6 carbon atoms. Representative examples include, but are not limited to, ethyl, n-propyl, isopropyl, butyl, isobutyl, sec-butyl, tert-butyl, n-pentyl, isopentyl, neopentyl, tert-pentyl, n-hexyl, isohexyl and the like.

The term “C₁₋₆alkoxy” in the present context designates a group —O—C₁₋₆-alkyl wherein C₁₋₆-alkyl is as defined above. Representative examples include, but are not limited to, methoxy, ethoxy, n-propoxy, isopropoxy, butoxy, isobutoxy, sec-butoxy, tert-butoxy, n-pentoxy, isopentoxy, neopentoxy, tert-pentoxy, n-hexoxy, isohexoxy and the like.

The term “C₂₋₆-alkenyl” as used herein, represent an olefinically unsaturated branched or straight hydrocarbon group having from 2 to 6 carbon atoms and at least one double bond. Examples of such groups include, but are not limited to, vinyl, 1-propenyl, 2-propenyl, allyl, iso-propenyl, 1,3-butadienyl, 1-butenyl, hexenyl, pentenyl and the like.

The term “C₃₋₁₀-cycloalkyl” as used herein represents a saturated mono-, bi-, tri- or spi-rocarbocyclic group having from 3 to 10 carbon atoms. Representative examples are cyclopropyl, cyclobutyl, cyclopentyl, cyclohexyl, cycloheptyl, cyclooctyl, cyclononyl, cyclodecyl, bicyclo[3.2.1]octyl, spiro[4.5]decyl, norpinyl, norbonyl, norcaryl, adamantyl and the like.

The term “C₃₋₈-heterocyclyl” as used herein represents a saturated 3 to 8 membered ring containing one or more heteroatoms selected from nitrogen, oxygen and sulfur. Representative examples are pyrrolidyl, piperidyl, piperazinyl, morpholinyl, thiomorpholinyl, aziridinyl, tetrahydrofuranyl and the like.

The term “aryl” as used herein represents a carbocyclic aromatic ring system being either monocyclic, bicyclic, or polycyclic, such as phenyl, biphenyl, naphthyl, anthracenyl, phenanthrenyl, fluorenyl, indenyl, pentalenyl, azulenyl, biphenylenyl and the like. Aryl is also intended to include the partially hydrogenated derivatives of the carbocyclic aromatic systems enumerated above. Non-limiting examples of such partially hydrogenated derivatives are 1,2,3,4-tetrahydronaphthyl, 1,4-dihydronaphthyl and the like.

The term “heteroaryl” as used herein represents a heterocyclic aromatic ring system containing one or more heteroatoms selected from nitrogen, oxygen and sulfur such as furyl, thienyl, pyrrolyl, oxazolyl, thiazolyl, imidazolyl, isoxazolyl, isothiazolyl, 1,2,3-triazolyl, 1,2,4-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4- thiadiazolyl, tetrazolyl, thiadiazinyl, indolyl, isoindolyl, benzofuranyl, benzothiophenyl (thianaphthenyl), indazolyl, benzimidazolyl, benzthiazolyl, benzisothiazolyl, benzoxazolyl, benzisoxazolyl, purinyl, quinazolinyl, quinolizinyl, quinolinyl, isoquinolinyl, quinoxalinyl, naphthyridinyl, pteridinyl, carbazolyl, azepinyl, diazepinyl, acridinyl and the like. Heteroaryl is also intended to include the partially hydrogenated derivatives of the heterocyclic systems enumerated above. Non-limiting examples of such partially hydrogenated derivatives are 2,3-dihydrobenzofuranyl, 3,4-dihydroisoquinolinyl, pyrrolinyl, pyrazolinyl, indolinyl, oxazolidinyl, oxazolinyl, oxazepinyl and the like.

The term “perhalomethyl” as used herein designates a methyl moiety substituted with three halogen atoms. Non-limiting examples of perhalomethyl are CF₃, CCl₃, and CF₂Cl.

The term “perhalomethoxy” as used herein designates a perhalomethyl linked via an oxygen atom, e.g. —O—CF₃, —O—CCl₃, and —O—CF₂Cl

The term “ring system” as used herein includes aromatic as well as non-aromatic ring moieties, which may be monocyclic, bicyclic or polycyclic, and they encompass moieties with zero, one or more hetereatoms selected from nitrogen, oxygen and sulphur. Non-limiting examples of such ring systems are aryl, C₃₋₈-heterocyclyl and heteroaryl.

The term “heterocyclic system” as used herein includes aromatic as well as non-aromatic ring moieties, which may be monocyclic, bicyclic or polycyclic, and containing in their ring structure one or more heteroatoms selected from nitrogen, oxygen and sulfur. Non-limiting examples of such heterocyclic systems are C₃₋₈-heterocyclyl and heteroaryl.

Certain of the above defined terms may occur more than once in the structural formulae, and upon such occurrence each term shall be defined independently of the other.

The term “optionally substituted” as used herein means that the groups in question are either unsubstituted or substituted with one or more of the substituents specified. When the groups in question are substituted with more than one substituent the substituents may be the same or different.

The terms “disease”, “condition” and “disorder” as used herein are used interchangeably to specify a state of a patient which is not the normal physiological state of man.

The term “treatment” as used herein means the management and care of a patient having developed a disease, condition or disorder, as well as the management and care of an individual at risk of developing the disease, condition or disorder prior to the clinical onset of said disease, condition or disorder. The purpose of treatment is to combat the disease, condition or disorder, as well as to to combat the development of the disease, condition or disorder. Treatment includes the administration of the active compounds to prevent or delay the onset of the symptoms or complications and to eliminate or control the disease, condition or disorder as well as to alleviate the symptoms or complications associated with the disease, condition or disorder.

The term “effective amount” as used herein means a dosage which is sufficient in order for the treatment of the patient to be effective compared with no treatment.

The term “modulate” as used herein means to influence, i.e. to modulate a parameter means to influence that parameter in a desired way. Examples are to modulate insulin secretion from beta cells and to modulate the plasma level of free fatty acids.

The term “medicament” as used herein means a pharmaceutical composition suitable for administration of the pharmaceutically active compound to a patient.

The term “pharmaceutically acceptable” as used herein means suited for normal pharmaceutical applications, i.e. giving rise to no adverse events in patients etc.

### DESCRIPTION OF THE INVENTION

In one aspect the present invention relates to a compound of the general formula (I): 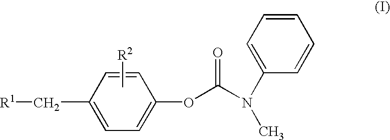
wherein
R¹ is selected from hydroxy, sulfanyl, sulfo, C₁₋₆-alkyl, C₁₋₆alkoxy, C₂₋₆-alkenyl, aryl, heteroaryl, C₃₋₈-heterocyclyl and C₃₋₁₀-cycloalkyl, wherein each of hydroxy, sulfanyl, sulfo, C₁₋₆alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, C₁₋₆alkyl, C₁₋₆alkoxy, C₂₋₆-alkenyl, aryl, heteroaryl, C₃₋₈-heterocyclyl, and C₃₋₁₀-cycloalkyl, wherein each of hydroxy, sulfanyl, sulfo, C₁₋₆alkyl, C₁₋₆alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆alkyl, C₁₋₆alkoxy, C₂₋₆-alkenyl, aryl, heteroaryl, C₃₋₈-heterocyclyl, and C₃₋₁₀-cycloalkyl;
R² is selected from hydrogen, hydroxy, sulfanyl, amino, halogen, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, C₁₋₆-alkyl, C₁₋₆-alkoxy, C₂₋₆-alkenyl, aryl, heteroaryl, C₃₋₈-heterocyclyl, and C₃₋₁₀-cycloalkyl, wherein each of hydroxy, sulfanyl, amino, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;
with the proviso that said compound is not
Methyl-phenyl-carbamic acid 4-(3-chloro-5-trifluoromethyl)-pyridine-2-ylmethyl)-phenyl ester,
Methyl-phenyl-carbamic acid 4-(1,3,5-trimethyl-1H-pyrazol-4-ylmethyl)-phenyl ester,
Methyl-phenyl-carbamic acid 4-(2-cyano-ethyl)-phenyl ester,
Methyl-phenyl-carbamic acid 4-pentyl-phenyl ester,
Methyl-phenyl-carbamic acid 4-(2-methoxy-ethyl)-phenyl ester,
Methyl-phenyl-carbamic acid 4-[2-(4-chloro-phenyl)-ethyl]-phenyl ester,
Methyl-phenyl-carbamic acid 4-(4-hydroxy-benzyl)-phenyl ester,{2-[4-(Methyl-phenyl-carbamoyloxy)-phenyl]-ethyl}-carbamic acid tert-butyl ester,
Methyl-phenyl-carbamic acid 4-(2-amino-ethyl)phenyl ester,
Methyl-phenyl-carbamic acid 4-[2-(toluene-4-sulfonylamino)-ethyl]-phenyl ester,
Methyl-phenyl-carbamic acid 4-[2-(5-dimethylamino-naphthalene-1-sulfonylamino)-ethyl]-phenyl ester,
Methyl-phenyl-carbamic acid 4-[2-(3,4-difluoro-benzenesulfonylamino)-ethyl]-phenyl ester, 2-{2-[4-(Methyl-phenyl-carbamoyloxy)-phenyl]-ethylsulfamoyl}-benzoic acid methyl ester,
Methyl-phenyl-carbamic acid 4-[2-(2,5-dichloro-thiophene-3-sulfonylamino)-ethyl]-phenyl ester,
Methyl-phenyl-carbamic acid 4-[2-(5-pyridin-2-yl-thiophene-2-sulfonylamino)-ethyl]-phenyl ester,
Methyl-phenyl-carbamic acid 4-[2-(1-methyl-1H-imidazole-4-sulfonylamino)-ethyl]-phenyl ester,
Methyl-phenyl-carbamic acid 4-[2-(5-chloro-1,3-dimethyl-1H-pyrazole-4-sulfonylamino)-ethyl]-phenyl,
Methyl-phenyl-carbamic acid 4-[2-(4-nitro-benzenesulfonylamino)-ethyl]-phenyl ester,
Methyl-phenyl-carbamic acid 4-[2-(6-chloro-imidazo[2,1-b]thiazole-5-sulfonylamino)-ethyl]-phenyl ester,
Methyl-phenyl-carbamic acid 4-[2-(2-trifluoromethoxy-benzenesulfonylamino)-ethyl]-phenyl ester,
Methyl-phenyl-carbamic acid 4-(2-dimethylaminomethanesulfonylamino-ethyl)-phenyl ester,
Methyl-phenyl-carbamic acid 4-(2-methanesulfonylamino-ethyl)-phenyl ester,
Methyl-phenyl-carbamic acid 4-[2-(6-morpholin-4-yl-pyridine-3-sulfonylamino)-ethyl]-phenyl ester,
Methyl-phenyl-carbamic acid 4-[2-(6-phenoxy-pyridine-3-sulfonylamino)-ethyl]-phenyl ester,
Methyl-phenyl-carbamic acid 4-{2-[4-(4-methyl-piperazin-1-yl)-benzenesulfonylamino]-ethyl}-phenyl ester,
Methyl-phenyl-carbamic acid 4-[2-(4-dimethylamino-benzenesulfonylamino)-ethyl]-phenyl ester,
Methyl-phenyl-carbamic acid 4-{2-[4-(2-pyrrolidin-1-yl-ethoxy)-benzenesulfonylamino]-ethyl}-phenyl ester,
Methyl-phenyl-carbamic acid 4-pyridin-2-ylmethyl-phenyl ester,
Methyl-phenyl-carbamic acid 4-pyridin-3-ylmethyl-phenyl ester,
Methyl-phenyl-carbamic acid 4-(4-trifluoromethyl-benzyl)-phenyl ester,
Methyl-phenyl-carbamic acid 4-thiophen-3-ylmethyl-phenyl ester,
Methyl-phenyl-carbamic acid 4-thiophen-2-ylmethyl-phenyl ester,
Methyl-phenyl-carbamic acid 4-[2-(4-amino-benzenesulfonylamino)-ethyl]-phenyl ester,
Methyl-phenyl-carbamic acid 4-{2-[(pyridine-3-carbonyl)-amino]-ethyl}-phenyl ester,
Methyl-phenyl-carbamic acid 4-[2-(2-dimethylamino-acetylamino)-ethyl]-phenyl ester,
Methyl-phenyl-carbamic acid 2-(toluene-4-sulfonyl)-1,2,3,4-tetrahydro-isoquinolin-7-yl ester,
Methyl-phenyl-carbamic acid 4-[4-(2-pyrrolidin-1-yl-ethoxy)-benzyl]-phenyl ester,
Methyl-phenyl-carbamic acid 4-{2-[(1-methyl-piperidine-4-carbonyl)-amino]-ethyl}-phenyl ester,
3,3-Dimethyl4-{2-[4-(methyl-phenyl-carbamoyloxy)-phenyl]-ethylcarbamoyl)-butyric acid,
Methyl-phenyl-carbamic acid 4-{2-[4-(4-methyl-piperazin-1-yl)-benzoylamino]-ethyl}-phenyl ester,
Methyl-phenyl-carbamic acid 4-{2-[4-(4-methyl-piperazin-1-ylmethyl)-benzoylamino]-ethyl}-phenyl ester,
Methyl-phenyl-carbamic acid 4-[2-(4,4-dimethyl-2,6-dioxo-piperidin-1-yl)-ethyl]-phenyl ester,
3,3-Dimethyl-4-{2-[4-(methyl-phenyl-carbamoyloxy)-phenyl]-ethylcarbamoyl}-butyric acid ethyl ester,
Methyl-phenyl-carbamic acid 4-hydroxymethyl-phenyl ester,
Methyl-phenyl-carbamic acid 4-(2-hydroxy-ethyl)-phenyl ester,
Methyl-phenyl-carbamic acid 4-(4-dimethylamino-pyridin-2-ylmethyl)-phenyl ester,
Methyl-phenyl-carbamic acid 4-(4-imidazol-1-yl-phenoxymethyl)-phenyl ester,
Methyl-phenyl-carbamic acid 4-[4-(2-dimethylamino-ethyl)-phenoxymethyl]-phenyl ester,
Methyl-phenyl-carbamic acid 4-(pyrazol-1-yloxymethyl)-phenyl ester,
Methyl-phenyl-carbamic acid 4-(imidazol-1-yloxymethyl)-phenyl ester,
Methyl-phenyl-carbamic acid 4-(2-oxo-2H-pyridin-1-ylmethyl)-phenyl ester,
Methyl-phenyl-carbamic acid 4-[2-(pyridin-2-yloxy)-ethyl]-phenyl ester,
Methyl-phenyl-carbamic acid 4-[2-(4-imidazol-1-yl-phenoxy)-ethyl]-phenyl ester,
Methyl-phenyl-carbamic acid 4-{2-[4-(2-dimethylamino-ethyl)-phenoxy]-ethyl}-phenyl ester,
Methyl-phenyl-carbamic acid 4-[2-(pyrazol-1-yloxy)-ethyl]-phenyl ester,
Methyl-phenyl-carbamic acid 4-[2-(imidazol-1-yloxy)-ethyl]-phenyl ester,
Methyl-phenyl-carbamic acid 4-(5-methyl-pyridin-2-ylmethyl)-phenyl ester,
Methyl-phenyl-carbamic acid 4-(4-oxo-4H-pyridin-1-ylmethyl)-phenyl ester,
Methyl-phenyl-carbamic acid 4-[2-(pyridin-3-yloxy)-ethyl]-phenyl ester,
Methyl-phenyl-carbamic acid 4-(2-oxo-2H-pyridin-1-ylmethyl)-phenyl ester,
Methyl-phenyl-carbamic acid 4-(pyridin-3-yloxymethyl)-phenyl ester,
Methyl-phenyl-carbamic acid 4-[2-(2,5-dioxo-pyrrolidin-1-yl)-ethyl]-phenyl ester,
Methyl-phenyl-carbamic acid 4-[2-(1,3-dioxo-1,3-dihydro-pyrrolo[3,4-]pyridin-2-yl)-ethyl]-phenyl ester,
Methyl-phenyl-carbamic acid 4-(1-methyl-1H-imidazol-2-ylsulfanylmethyl)-phenyl ester,
Methyl-phenyl-carbamic acid 4-tetrazol-1-ylmethyl-phenyl ester,
Methyl-phenyl-carbamic acid 4-(2,5-dioxo-pyrrolidin-1-ylmethyl)-phenyl ester,Methyl-phenyl-carbamic acid 4-[2-(2-thioxo-2H-pyridin-1-yl)-ethyl]-phenyl ester,
Methyl-phenyl-carbamic acid 4-(1,3-dioxo-1,3-dihydro-pyrrolo[3,4)pyridin-2-ylmethyl)-phenyl ester,
Methyl-phenyl-carbamic acid 4-[1,2,4]triazol-1-ylmethyl-phenyl ester,
Methyl-phenyl-carbamic acid 4-(2-thioxo-2H-pyridin-1-ylmethyl)-phenyl ester,Methyl-phenyl-carbamic acid 4-[2-(1-methyl-1H-imidazol-2-ylsulfanyl)-ethyl]-phenyl ester,ethyl-phenyl-carbamic acid 4-(2-tetrazol-1-yl-ethyl)-phenyl ester,Methyl-phenyl-carbamic acid 4-[2-(pyrimidin-2-yloxy)-ethyl]-phenyl ester, Methyl-phenyl-carbamic acid 4-[2-(pyridin-4-ylsulfanyl)-ethyl]-phenyl ester,Methyl-phenyl-carbamic acid 4-[2-(1-pyridin-3-yl-1H-imidazol-2-ylsulfanyl)-ethyl]-phenyl ester,Methyl-phenyl-carbamic acid 4-[2-(1,3-dioxo-1,3-dihydro-isoindol-2-yl)-ethyl]-phenyl ester, andMethyl-phenyl-carbamic acid 4-benzyl-phenyl ester;
as well as diastereomers, enantiomers or tautomeric forms thereof including mixtures of these, pharmaceutically acceptable salts thereof, pharmaceutically acceptable solvates thereof, or polymorphs.

In another aspect the present invention relates to a compound of the general formula (I): 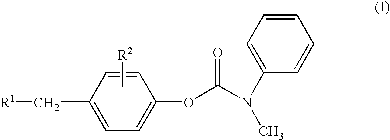
wherein
R¹ is selected from hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, C₁₋₆-alkyl, C₁₋₆-alkoxy, C₂₋₆-alkenyl, aryl, heteroaryl, C₃₋₈-heterocyclyl, and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;
R² is selected from hydrogen, hydroxy, sulfanyl, amino, halogen, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, C₁₋₆alkyl, C₁₋₆-alkoxy, C₂₋₆-alkenyl, aryl, heteroaryl, C₃₋₈-heterocyclyl, and C₃₋₁₀-cycloalkyl, wherein each of hydroxy, sulfanyl, amino, sulfo, C₁₋₆-alkyl, C₁₋₆alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;
with the proviso that said compound is not
Methyl-phenyl-carbamic acid 4-(3-chloro-5-trifluoromethyl)-pyridine-2-ylmethyl)-phenyl ester,
Methyl-phenyl-carbamic acid 4-(1,3,5-trimethyl-1H-pyrazol-4-ylmethyl)-phenyl ester,
Methyl-phenyl-carbamic acid 4-(2-cyano-ethyl)-phenyl ester,
Methyl-phenyl-carbamic acid 4-pentyl-phenyl ester,
Methyl-phenyl-carbamic acid 4-(2-methoxy-ethyl)-phenyl ester,
Methyl-phenyl-carbamic acid 4-[2-(4-chloro-phenyl)-ethyl]-phenyl ester,
Methyl-phenyl-carbamic acid 4-(4-hydroxy-benzyl)-phenyl ester,{2-[4-(Methyl-phenyl-carbamoyloxy)-phenyl]-ethyl}-carbamic acid tert-butyl ester,
Methyl-phenyl-carbamic acid 4-(2-amino-ethyl)phenyl ester,
Methyl-phenyl-carbamic acid 4-[2-(toluene-4-sulfonylamino)-ethyl]-phenyl ester,
Methyl-phenyl-carbamic acid 4-[2-(5-dimethylamino-naphthalene-1-sulfonylamino)-ethyl]-phenyl ester,
Methyl-phenyl-carbamic acid 4-[2-(3,4-difluoro-benzenesulfonylamino)-ethyl]-phenyl ester,
2-{2-[4-(Methyl-phenyl-carbamoyloxy)-phenyl]-ethylsulfamoyl}-benzoic acid methyl ester,
Methyl-phenyl-carbamic acid 4-[2-(2,5-dichloro-thiophene-3-sulfonylamino)-ethyl]-phenyl ester,
Methyl-phenyl-carbamic acid 4-[2-(5-pyridin-2-yl-thiophene-2-sulfonylamino)-ethyl]-phenyl ester,
Methyl-phenyl-carbamic acid 4-[2-(1-methyl-1H-imidazole-4-sulfonylamino)-ethyl]-phenyl ester,
Methyl-phenyl-carbamic acid 4-[2-(5-chloro-1,3-dimethyl-1H-pyrazole-4-sulfonylamino)-ethyl]-phenyl,
Methyl-phenyl-carbamic acid 4-[2-(4-nitro-benzenesulfonylamino)-ethyl]-phenyl ester,
Methyl-phenyl-carbamic acid 4-[2-(6-chloro-imidazo[2,1-b]thiazole-5-sulfonylamino)-ethyl]-phenyl ester,
Methyl-phenyl-carbamic acid 4-[2-(2-trifluoromethoxy-benzenesulfonylamino)-ethyl]-phenyl ester,
Methyl-phenyl-carbamic acid 4-(2-dimethylaminomethanesulfonylamino-ethyl)-phenyl ester,
Methyl-phenyl-carbamic acid 4-(2-methanesulfonylamino-ethyl)-phenyl ester,
Methyl-phenyl-carbamic acid 4-[2-(6-morpholin-4-yl-pyridine-3-sulfonylamino)-ethyl]-phenyl ester,
Methyl-phenyl-carbamic acid 4-[2-(6-phenoxy-pyridine-3-sulfonylamino)-ethyl]-phenyl ester,
Methyl-phenyl-carbamic acid 4-{2-[4-(4-methyl-piperazin-1-yl)-benzenesulfonylamino]-ethyl}-phenyl ester,
Methyl-phenyl-carbamic acid 4-[2-(4-dimethylamino-benzenesulfonylamino)-ethyl]-phenyl ester,
Methyl-phenyl-carbamic acid 4-{2-[4-(2-pyrrolidin-1-yl-ethoxy)-benzenesulfonylamino]-ethyl}-phenyl ester,
Methyl-phenyl-carbamic acid 4-pyridin-2-ylmethyl-phenyl ester,
Methyl-phenyl-carbamic acid 4-pyridin-3-ylmethyl-phenyl ester,
Methyl-phenyl-carbamic acid 4-(4-trifluoromethyl-benzyl)-phenyl ester,
Methyl-phenyl-carbamic acid 4-thiophen-3-ylmethyl-phenyl ester,
Methyl-phenyl-carbamic acid 4-thiophen-2-ylmethyl-phenyl ester,
Methyl-phenyl-carbamic acid 4-[2-(4-amino-benzenesulfonylamino)-ethyl]-phenyl ester,
Methyl-phenyl-carbamic acid 4-{2-[(pyridine-3-carbonyl)-amino]-ethyl}-phenyl ester,
Methyl-phenyl-carbamic acid 4-[2-(2-dimethylamino-acetylamino)-ethyl]-phenyl ester,
Methyl-phenyl-carbamic acid 2-(toluene-4-sulfonyl)-1,2,3,4-tetrahydro-isoquinolin-7-yl ester,
Methyl-phenyl-carbamic acid 4-[4-(2-pyrrolidin-1-yl-ethoxy)-benzyl]-phenyl ester,
Methyl-phenyl-carbamic acid 4-{2-[(1-methyl-piperidine-4-carbonyl)-amino]-ethyl}-phenyl ester,
3,3-Dimethyl-4-{2-[4-(methyl-phenyl-carbamoyloxy)-phenyl]-ethylcarbamoyl}-butyric acid,
Methyl-phenyl-carbamic acid 4-{2-[4-(4-methyl-piperazin-1-yl)-benzoylamino]-ethyl}-phenyl ester,
Methyl-phenyl-carbamic acid 4-{2-[4-(4-methyl-piperazin-1-ylmethyl)-benzoylamino]-ethyl}-phenyl ester,
Methyl-phenyl-carbamic acid 4-[2-(4,4-dimethyl-2,6-dioxo-piperidin-1-yl)-ethyl]-phenyl ester,
3,3-Dimethyl-4-{2-[4-(methyl-phenyl-carbamoyloxy)-phenyl]-ethylcarbamoyl}-butyric acid ethyl ester,
Methyl-phenyl-carbamic acid 4-hydroxymethyl-phenyl ester,
Methyl-phenyl-carbamic acid 4-(2-hydroxy-ethyl)-phenyl ester,
Methyl-phenyl-carbamic acid 4-(4-dimethylamino-pyridin-2-ylmethyl)-phenyl ester,
Methyl-phenyl-carbamic acid 4-(4-imidazol-1-yl-phenoxymethyl)-phenyl ester,
Methyl-phenyl-carbamic acid 4-[4-(2-dimethylamino-ethyl)-phenoxymethyl]-phenyl ester,
Methyl-phenyl-carbamic acid 4-(pyrazol-1-yloxymethyl)-phenyl ester,
Methyl-phenyl-carbamic acid 4-(imidazol-1-yloxymethyl)-phenyl ester,
Methyl-phenyl-carbamic acid 4-(2-oxo-2H-pyridin-1-ylmethyl)-phenyl ester,
Methyl-phenyl-carbamic acid 4-[2-(pyridin-2-yloxy)-ethyl]-phenyl ester,
Methyl-phenyl-carbamic acid 4-[2-(4-imidazol-1-yl-phenoxy)-ethyl]-phenyl ester,
Methyl-phenyl-carbamic acid 4-{2-[4-(2-dimethylamino-ethyl)-phenoxy]-ethyl}-phenyl ester,
Methyl-phenyl-carbamic acid 4-[2-(pyrazol-1-yloxy)-ethyl]-phenyl ester,
Methyl-phenyl-carbamic acid 4-[2-(imidazol-1-yloxy)-ethyl]-phenyl ester,
Methyl-phenyl-carbamic acid 4-(5-methyl-pyridin-2-ylmethyl)-phenyl ester,
Methyl-phenyl-carbamic acid 4-(4-oxo-4H-pyridin-1-ylmethyl)-phenyl ester,
Methyl-phenyl-carbamic acid 4-[2-(pyridin-3-yloxy)-ethyl]-phenyl ester,
Methyl-phenyl-carbamic acid 4-(2-oxo-2H-pyridin-1-ylmethyl)-phenyl ester,
Methyl-phenyl-carbamic acid 4-(pyridin-3-yloxymethyl)-phenyl ester,
Methyl-phenyl-carbamic acid 4-[2-(2,5-dioxo-pyrrolidin-1-yl)-ethyl]-phenyl ester,
Methyl-phenyl-carbamic acid 4-[2-(1,3-dioxo-1,3-dihydro-pyrrolo[3,4-]pyridin-2-yl)-ethyl]-phenyl ester,
Methyl-phenyl-carbamic acid 4-(1-methyl-1H-imidazol-2-ylsulfanylmethyl)-phenyl ester,
Methyl-phenyl-carbamic acid 4-tetrazol-1-ylmethyl-phenyl ester,
Methyl-phenyl-carbamic acid 4-(2,5-dioxo-pyrrolidin-1-ylmethyl)-phenyl ester,Methyl-phenyl-carbamic acid 4-[2-(2-thioxo-2H-pyridin-1-yl)-ethyl]-phenyl ester,
Methyl-phenyl-carbamic acid 4-(1,3-dioxo-1,3-dihydro-pyrrolo[3,4)pyridin-2-ylmethyl)-phenyl ester,
Methyl-phenyl-carbamic acid 4-[1,2,4]triazol-1-ylmethyl-phenyl ester,
Methyl-phenyl-carbamic acid 4-(2-thioxo-2H-pyridin-1-ylmethyl)-phenyl ester,Methyl-phenyl-carbamic acid 4-[2-(1-methyl-1H-imidazol-2-ylsulfanyl)-ethyl]-phenyl ester,ethyl-phenyl-carbamic acid 4-(2-tetrazol-1-yl-ethyl)-phenyl ester,Methyl-phenyl-carbamic acid 4-[2-(pyrimidin-2-yloxy)-ethyl]-phenyl ester, Methyl-phenyl-carbamic acid 4-[2-(pyridin-4-ylsulfanyl)-ethyl]-phenyl ester, Methyl-phenyl-carbamic acid 4-[2-(1-pyridin-3-yl-1H-imidazol-2-ylsulfanyl)-ethyl]-phenyl ester,Methyl-phenyl-carbamic acid 4-[2-(1,3-dioxo-1,3-dihydro-isoindol-2-yl)-ethyl]-phenyl ester, andMethyl-phenyl-carbamic acid 4-benzyl-phenyl ester;
as well as diastereomers, enantiomers or tautomeric forms thereof including mixtures of these, pharmaceutically acceptable salts thereof, pharmaceutically acceptable solvates thereof, or polymorphs.

In another aspect the present invention relates to a compound of the general formula (I): 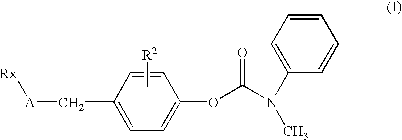
wherein
Rx is selected from aryl, heteroaryl, C₃₋₈-heterocyclyl and C₃₋₁₀-cycloalkyl, wherein each of aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, C₁₋₆-alkyl, C₁₋₆-alkoxy, C₂₋₆-alkenyl, aryl, heteroaryl, C₃₋₈-heterocyclyl, and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C_{3- 10}-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;
A is selected from —CH₂O—, —CH₂S—, —S—, —O—, —S(═O)₂—, —CH₂(S═O)₂, and —CH₂— or is absent;
R² is selected from hydrogen, hydroxy, sulfanyl, amino, halogen, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, C₁₋₆-alkyl, C₁₋₆-alkoxy, C₂₋₆-alkenyl, aryl, heteroaryl, C₃₋₈-heterocyclyl, and C₃₋₁₀-cycloalkyl, wherein each of hydroxy, sulfanyl, amino, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;
with the proviso that said compound is not
Methyl-phenyl-carbamic acid 4-(3-chloro-5-trifluoromethyl)-pyridine-2-ylmethyl)-phenyl ester,
Methyl-phenyl-carbamic acid 4-(1,3,5-trimethyl-1H-pyrazol-4-ylmethyl)-phenyl ester,
Methyl-phenyl-carbamic acid 4-[2-(1,3-dioxo-1,3-dihydro-pyrrolo[3,4-]pyridin-2-yl)-ethyl]-phenyl ester,
Methyl-phenyl-carbamic acid 4-(1,3-dioxo-1,3-dihydro-pyrrolo[3,4)pyridin-2-ylmethyl)-phenyl ester,
Methyl-phenyl-carbamic acid 4-[2-(1,3-dioxo-1,3-dihydro-isoindol-2-yl)-ethyl]-phenyl ester,
Methyl-phenyl-carbamic acid 4-[2-(4-chloro-phenyl)-ethyl]-phenyl ester,
Methyl-phenyl-carbamic acid 4-(4-hydroxy-benzyl)-phenyl ester,
Methyl-phenyl-carbamic acid 4-pyridin-2-ylmethyl-phenyl ester,
Methyl-phenyl-carbamic acid 4-pyridin-3-ylmethyl-phenyl ester,
Methyl-phenyl-carbamic acid 4-(4-trifluoromethyl-benzyl)-phenyl ester,
Methyl-phenyl-carbamic acid 4-thiophen-3-ylmethyl-phenyl ester,
Methyl-phenyl-carbamic acid 4-thiophen-2-ylmethyl-phenyl ester,
Methyl-phenyl-carbamic acid 4-[4-(2-pyrrolidin-1-yl-ethoxy)-benzyl]-phenyl ester,
Methyl-phenyl-carbamic acid 4-(4-dimethylamino-pyridin-2-ylmethyl)-phenyl ester,
Methyl-phenyl-carbamic acid 4-(4-imidazol-1-yl-phenoxymethyl)-phenyl ester,
Methyl-phenyl-carbamic acid 4-(pyrazol-1-yloxymethyl)-phenyl ester,
Methyl-phenyl-carbamic acid 4-(imidazol-1-yloxymethyl)-phenyl ester,
Methyl-phenyl-carbamic acid 4-[2-(pyridin-2-yloxy)-ethyl]-phenyl ester,
Methyl-phenyl-carbamic acid 4-[2-(4-imidazol-1-yl-phenoxy)-ethyl]-phenyl ester,
Methyl-phenyl-carbamic acid 4-[2-(pyrazol-1-yloxy)-ethyl]-phenyl ester,
Methyl-phenyl-carbamic acid 4-[2-(imidazol-1-yloxy)-ethyl]-phenyl ester,
Methyl-phenyl-carbamic acid 4-(5-methyl-pyridin-2-ylmethyl)-phenyl ester,
Methyl-phenyl-carbamic acid 4-(4-oxo-4H-pyridin-1-ylmethyl)-phenyl ester,
Methyl-phenyl-carbamic acid 4-[2-(pyridin-3-yloxy)-ethyl]-phenyl ester,
Methyl-phenyl-carbamic acid 4-(2-oxo-2H-pyridin-1-ylmethyl)-phenyl ester,
Methyl-phenyl-carbamic acid 4-(pyridin-3-yloxymethyl)-phenyl ester,
Methyl-phenyl-carbamic acid 4-[2-(2,5-dioxo-pyrrolidin-1-yl)-ethyl]-phenyl ester,
Methyl-phenyl-carbamic acid 4-(1-methyl-1H-imidazol-2-ylsulfanylmethyl)-phenyl ester,
Methyl-phenyl-carbamic acid 4-tetrazol-1-ylmethyl-phenyl ester,
Methyl-phenyl-carbamic acid 4-(2,5-dioxo-pyrrolidin-1-ylmethyl)-phenyl ester,Methyl-phenyl-carbamic acid 4-[1,2,4]triazol-1-ylmethyl-phenyl ester,
Methyl-phenyl-carbamic acid 4-[2-(1-methyl-1H-imidazol-2-ylsulfanyl)-ethyl]-phenyl ester,ethyl-phenyl-carbamic acid 4-(2-tetrazol-1-yl-ethyl)-phenyl ester,Methyl-phenyl-carbamic acid 4-[2-(pyrimidin-2-yloxy)-ethyl]-phenyl ester, Methyl-phenyl-carbamic acid 4-[2-(pyridin-4-ylsulfanyl)-ethyl]-phenyl ester, Methyl-phenyl-carbamic acid 4-[2-(1-pyridin-3-yl-1H-imidazol-2-ylsulfanyl)-ethyl]-phenyl ester,
as well as diastereomers, enantiomers or tautomeric forms thereof including mixtures of these, pharmaceutically acceptable salts thereof, pharmaceutically acceptable solvates thereof, or polymorphs.

In another aspect the present invention relates to a compound of the general formula (I): 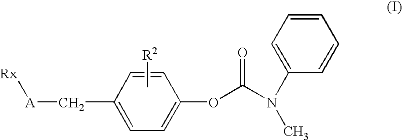
wherein
Rx is selected from aryl, heteroaryl, C₃₋₈-heterocyclyl, and C₃₋₁₀-cycloalkyl;
A is selected from —CH₂O—, —CH₂S—, —CH₂—;
R² is hydrogen
with the proviso that said compound is not
Methyl-phenyl-carbamic acid 4-[2-(pyridin-2-yloxy)-ethyl]-phenyl ester,
Methyl-phenyl-carbamic acid 4-[2-(pyrazol-1-yloxy)-ethyl]-phenyl ester,
Methyl-phenyl-carbamic acid 4-[2-(imidazol-1-yloxy)-ethyl]-phenyl ester,
Methyl-phenyl-carbamic acid 4-[2-(pyridin-3-yloxy)-ethyl]-phenyl ester, ethyl-phenyl-carbamic acid 4-(2-tetrazol-1-yl-ethyl)-phenyl ester,Methyl-phenyl-carbamic acid 4-[2-(pyrimidin-2-yloxy)-ethyl]-phenyl ester, Methyl-phenyl-carbamic acid 4-[2-(pyridin-4-ylsulfanyl)-ethyl]-phenyl ester,as well as diastereomers, enantiomers or tautomeric forms thereof including mixtures of these, pharmaceutically acceptable salts thereof, pharmaceutically acceptable solvates thereof, or polymorphs.

In another aspect the present invention relates to a compound of the general formula (I): 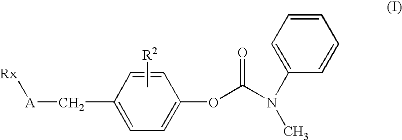
wherein
Rx is selected from aryl, heteroaryl, C₃₋₈-heterocyclyl, and C₃₋₁₀-cycloalkyl, wherein each of aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;
A is selected from —S—, —O—, —S(═O)₂—, —CH₂—,
R² is hydrogen
with the proviso that said compound is not
Methyl-phenyl-carbamic acid 4-(2-oxo-2H-pyridin-1-ylmethyl)-phenyl ester,
Methyl-phenyl-carbamic acid 4-[2-(1,3-dioxo-1,3-dihydro-pyrrolo[3,4-]pyridin-2-yl)-ethyl]-phenyl ester,
Methyl-phenyl-carbamic acid 4-[2-(1,3-dioxo-1,3-dihydro-isoindol-2-yl)-ethyl]-phenyl ester, andMethyl-phenyl-carbamic acid 4-[2-(4,4-dimethyl-2,6-dioxo-piperidin-1-yl)-ethyl]-phenyl ester,
Methyl-phenyl-carbamic acid 4-[2-(4-chloro-phenyl)-ethyl]-phenyl ester,
Methyl-phenyl-carbamic acid 4-(4-imidazol-1-yl-phenoxymethyl)-phenyl ester,
Methyl-phenyl-carbamic acid 4-(pyrazol-1-yloxymethyl)-phenyl ester,
Methyl-phenyl-carbamic acid 4-(imidazol-1-yloxymethyl)-phenyl ester,
Methyl-phenyl-carbamic acid 4-(pyridin-3-yloxymethyl)-phenyl ester,
Methyl-phenyl-carbamic acid 4-[2-(2,5-dioxo-pyrrolidin-1-yl)-ethyl]-phenyl ester,
Methyl-phenyl-carbamic acid 4-(1-methyl-1H-imidazol-2-ylsulfanylmethyl)-phenyl ester, ethyl-phenyl-carbamic acid 4-(2-tetrazol-1-yl-ethyl)-phenyl ester, as well as diastereomers, enantiomers or tautomeric forms thereof including mixtures of these, pharmaceutically acceptable salts thereof, pharmaceutically acceptable solvates thereof, or polymorphs.

In one embodiment the invention is concerned with compounds wherein R² is hydrogen.

In another embodiment the invention is concerned with compounds wherein R² is selected from hydrogen, hydroxy, sulfanyl, amino, halogen, sulfo, C₁₋₆alkyl, C₁₋₆-alkoxy, C₂₋₆-alkenyl, aryl, heteroaryl, C₁₋₆-heterocyclyl and C₃₋₁₀-cycloalkyl, wherein each of hydroxy, sulfanyl, amino, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, C₁₋₆-alkyl, C₁₋₆-alkoxy, C₂₋₆-alkenyl, perhalomethyl, and perhalomethoxy.

In another embodiment the invention is concerned with compounds wherein R² is selected from the group consisting of fluor, chlor, amino, —COOH, C₁₋₆-alkyl, C₁₋₆-alkoxy, and C₁₋₆-alkyl which has been substituted with one or more halogens.

In another embodiment the invention is concerned with compounds wherein R² is selected from the group consisting of

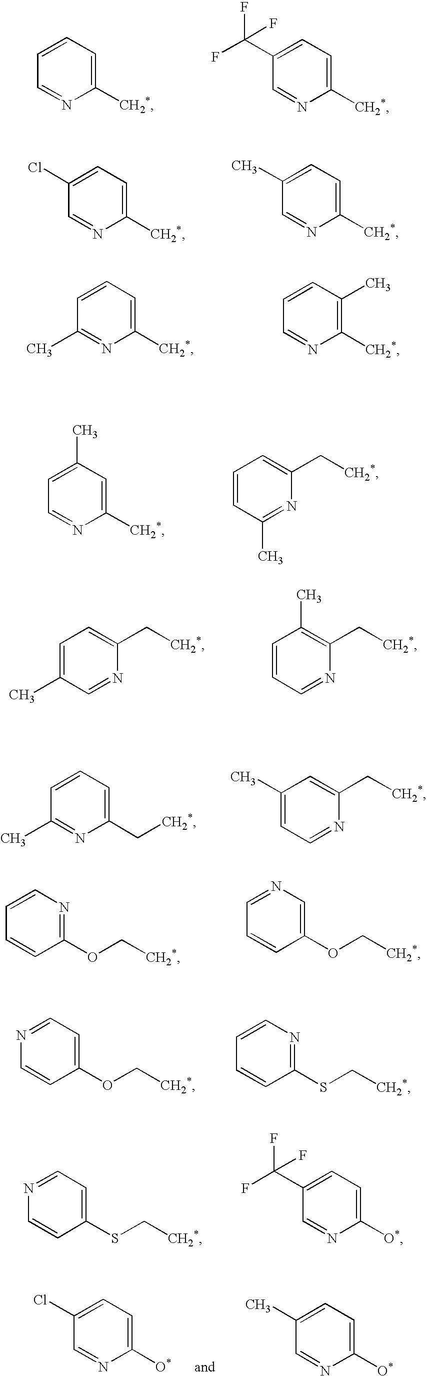

In another embodiment the invention is concerned with compounds wherein R² is selected from the group consisting of

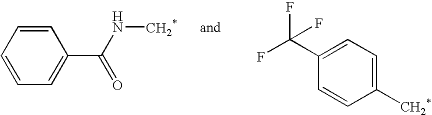

In another embodiment the invention is concerned with compounds wherein R² is selected from the group consisting of

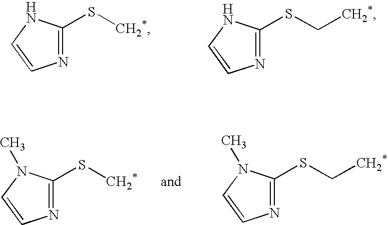

In another embodiment the invention is concerned with compounds wherein R² is selected from the group consisting of

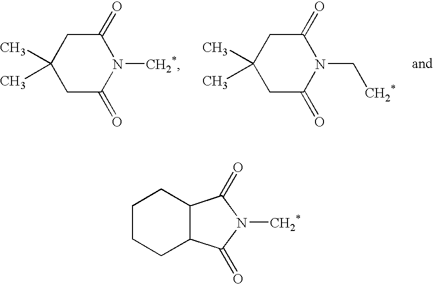

In another embodiment the invention is concerned with compounds wherein R¹ is selected from the group consisting of

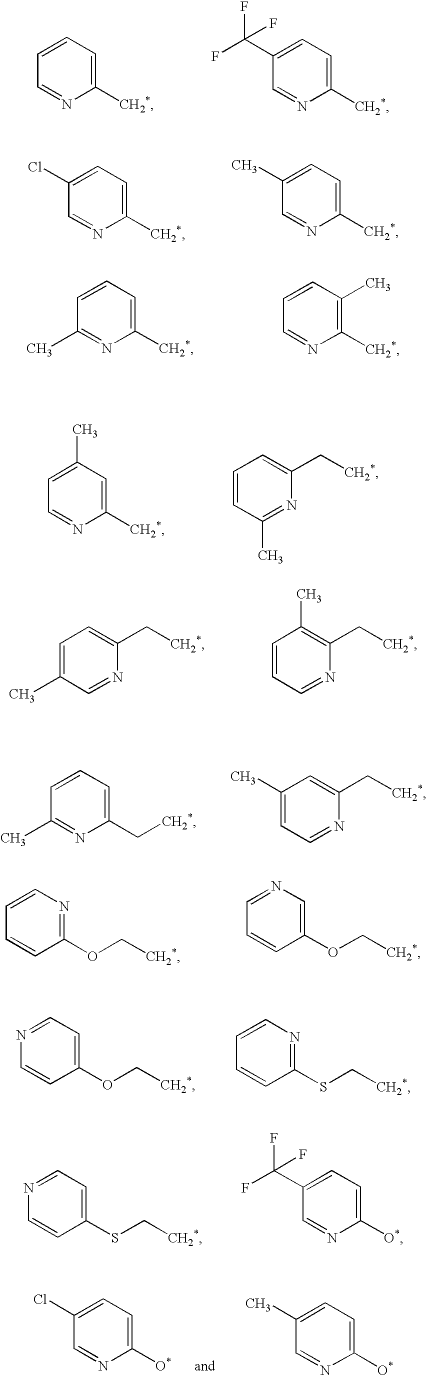

In another embodiment the invention is concerned with compounds wherein R² is selected from the group consisting of

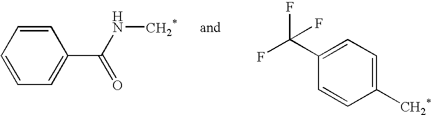

In another embodiment the invention is concerned with compounds wherein R² is selected from the group consisting of

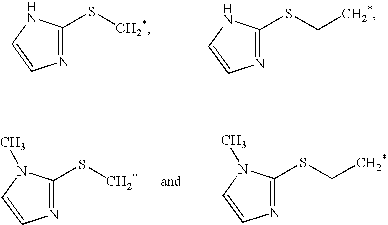

In another embodiment the invention is concerned with compounds wherein R² is selected from the group consisting of

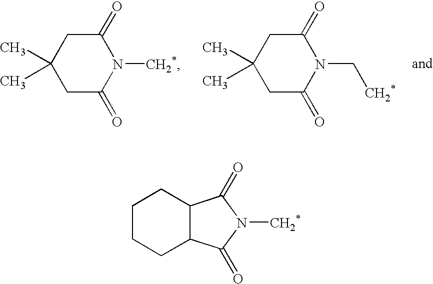

In another embodiment the invention is concerned with compounds wherein R² is hydrogen.

In another embodiment the invention is concerned with compounds wherein the sum of the molar weights of the substituents R¹ and R² is in the range from about 60 g/mole to about 250 g/mole.

In another embodiment the invention is concerned with compounds, having one free —COOH group.

In another embodiment the invention is concerned with compounds having one free amino group, or one monosubstituted amino group or one disubstituted amino group.

In another embodiment the invention is concerned with compounds having one substituted or unsubstituted pyridine ring.

In another embodiment the invention is concerned with compounds having one substituted or unsubstituted imidazole ring.

In another embodiment the invention is concerned with compounds wherein the molar weight of said compound is less than 650 g/mole.

The property cLog P of a compound which has no ionisable group is calculated using Sybyl 6.6 from Tripos Corporation, version 4.0 (provided by Biobyte Corp., Claremont Calif., USA).

In another embodiment the invention is concerned with compounds wherein the compound contains no ionisable group and wherein cLog P is in the range from 1.0 to 5.0.

In another embodiment the invention is concerned with compounds wherein the compound contains no ionisable group and wherein cLog P is in the range from 3.0 to 5.0.

A number of other properties of the compounds are calculated using Sybyl 6.6. from Tripos Corporation, i.e. the number of H-bond donors, the number of H-bond acceptors, the number of rotatable bonds. The polar surface area (PSA) is calculated using the SAVol program Based on SAVol 3.7 using Allinger vdw radii. Polar atoms are oxygens, nitrogens, plus hydrogens attached to O and N developed by R. S. Pearlman, J. M. Skell and F. Deanda, Laboratory for Molecular Graphics and Theoretical Modeling, College of Pharmacy, University of Texas, Austin, Tex. 78712, U.S.A.

In another embodiment the invention is concerned with compounds wherein the ACD LogD is in the range from 0.8 to 3.0.

In another embodiment the invention is concerned with compounds wherein the number of H-bond donors is 0, 1, or 2.

In another embodiment the invention is concerned with compounds wherein the number of H-bond donors is 0 or 1.

In another embodiment the invention is concerned with compounds wherein the number of H-bond acceptors is in the range from 4 to 9.

In another embodiment the invention is concerned with compounds wherein the number of H-bond acceptors is in the range from 4 to 7.

In another embodiment the invention is concerned with compounds wherein the number of H-bond acceptors is in the range from 4 to 6.

In another embodiment the invention is concerned with compounds wherein the number of rotatable bonds of said compound is in the range from 6 to 10.

In another embodiment the invention is concerned with compounds wherein the number of rotatable bonds of said compound is in the range from 8 to 10.

In another embodiment the invention is concerned with compounds wherein the polar surface area (PSA) is in the range from 30 Å² to120 Å².

In another embodiment the invention is concerned with compounds wherein the polar surface area (PSA) is in the range from 30 Å² to80 Å².

In another embodiment the invention is concerned with compounds wherein the polar surface area (PSA) is in the range from 30 Å² to 60 Å².

In another embodiment the invention is concerned with a compound selected from the group consisting of
Methyl-phenyl-carbamic acid 4-[2-(pyridin-4-yloxy)-ethyl]-phenyl ester,
Methyl-phenyl-carbamic acid 4-(1H-imidazol-2-ylsulfanylmethyl)-phenyl ester,
Methyl-phenyl-carbamic acid 4-[2-(1H-imidazol-2-ylsulfanyl)-ethyl]-phenyl ester,
Methyl-phenyl-carbamic acid 4-(6-methyl-pyridin-2-ylmethyl)-phenyl ester,
Methyl-phenyl-carbamic acid 4-(4-methyl-pyridin-2-ylmethyl)-phenyl ester,
Methyl-phenyl-carbamic acid 4-(3-methyl-pyridin-2-ylmethyl)-phenyl ester,
Methyl-phenyl-carbamic acid 4-[2-(6-methyl-pyridin-2-yl)-ethyl]-phenyl ester,
Methyl-phenyl-carbamic acid 4-[2-(5-methyl-pyridin-2-yl)-ethyl]-phenyl ester,
Methyl-phenyl-carbamic acid 4-[2-(4-methyl-pyridin-2-yl)-ethyl]-phenyl ester, and
Methyl-phenyl-carbamic acid 4-[2-(3-methyl-pyridin-2-yl)-ethyl]-phenyl ester.

In another aspect the present invention relates to a pharmaceutical composition comprising a compound of general formula I or a pharmaceutically acceptable salt thereof together with a pharmaceutically acceptable carrier or diluent.

In one embodiment the invention is concerned with a pharmaceutical composition in unit dosage form, comprising from about 0.05 to about 2000 mg, preferably from about 0.1 to about 500 mg and even more preferable from about 1.0 to about 100 mg of said compound according to the invention or pharmaceutically acceptable salt thereof.

In another embodiment the invention is concerned with a pharmaceutical composition for use as a medicament for inhibiting the lipolytic activity of hormone-sensitive lipase against triacylglycerols, diacylglycerols, cholesterol acyl esters or steroid acyl esters, said composition comprising a compound according to the invention or a pharmaceutically acceptable salt thereof together with a pharmaceutically acceptable carrier or diluent.

In another embodiment the invention is concerned with a pharmaceutical composition which is for oral administration.

In another embodiment the invention is concerned with a pharmaceutical composition which is for nasal, transdermal, pulmonal, or parenteral administration.

In another aspect the present invention relates to use of a compound according to the inventtion for the preparation of a pharmaceutical composition.

In one embodiment the invention is concerned with use of a compound according to the invention for inhibition of hormone sensitive lipase.

In another embodiment the invention is concerned with use of a compound according to the invention for preparation of a pharmaceutical composition for inhibition of the lipolytic activity of hormone-sensitive lipase against triacylglycerols, diacylglycerols, cholesterol acyl esters or steroid acyl esters.

In another embodiment the invention is concerned with use of a compound according to the invention for the preparation of a pharmaceutical composition for the treatment or prevention of any disorder where it is desirable to

modulate the plasma level of free fatty acids, glycerol, LDL-cholesterol, HDL-cholesterol, insulin and/or glucose; and/or

modulate intracellular triacylglycerol and cholesterol ester stores, intracellular level of fatty acids, fatty acid esters such as diacylglycerols, phosphatidic acids, long chain acyl-CoA's as well as citrate or malonyl-CoA; and/or

increase insulin sensitivity in adipose tissue, skeletal muscle, liver or pancreatic β cells; and/or modulate insulin secretion from pancreatic β cells.

In another embodiment the invention is concerned with the above use wherein said disorder is selected from the group consisting of insulin resistance, diabetes type 1, diabetes type 2, metabolic syndrome X, impaired glucose tolerance, hyperglycemia, dyslipidemia, obesity, atheroschlerosis, hypertension, abnormalities of lipoprotein metabolism and any combination thereof.

In another embodiment the invention is concerned with the use of a compound according to the invention for the preparation of a pharmaceutical composition for the treatment and/or prevention of dyslipidemia.

In another embodiment the invention is concerned with the use of a compound according to the invention for the preparation of a pharmaceutical composition for the treatment and/or prevention of hyperlipidemia.

In another embodiment the invention is concerned with the use of a compound according to the invention for the preparation of a pharmaceutical composition for the treatment and/or prevention of hyperglycemia.

In another embodiment the invention is concerned with the use of a compound according to the invention for lowering HbA_{1c}.

In another embodiment the invention is concerned with the preparation of a pharmaceutical composition for the treatment and/or prevention of diabetes type 2.

In another embodiment the invention is concerned with the use of a compound according to the invention for the preparation of a pharmaceutical composition for the treatment and/or prevention impaired glucose tolerance.

In another embodiment the invention is concerned with the use of a compound according to the invention for the preparation of a pharmaceutical composition for the treatment and/or prevention of metabolic syndrome X.

In another embodiment the invention is concerned with the use of a compound according to the invention for the preparation of a pharmaceutical composition for the treatment and/or prevention of atheroschlerosis.

In another embodiment the invention is concerned with the use of a compound according to the invention for the preparation of a pharmaceutical composition for delaying or prevention of the progression from impaired glucose tolerance to diabetes type 2.

In another embodiment the invention is concerned with the use of a compound according to the invention for the preparation of a pharmaceutical composition for delaying or prevention of the progression from non-insulin requiring diabetes type 2 to insulin requiring diabetes type 2.

In another embodiment the invention is concerned with the use according to above indicationns wherein a further antidiabetic, antiobesity, antihypertensive or appetite regulating drug is used.

In another embodiment the invention is concerned with the use according to above indications, wherein metformin is also used.

In another aspect the present invention is related to a method of treating a disorder of a patient where modulation of the activity of hormone-sensitive lipase is desired, the method comprising administering to a subject in need thereof a therapeutically effective amount of a compound according to the present invention or a pharmaceutically acceptable salt thereof.

In one embodiment the invention is concerned with a method of treating a disorder of a patient where lowering of the activity of hormone-sensitive lipase is desired, the method comprising administering to a subject in need thereof a therapeutically effective amount of a compound according to the invention or a pharmaceutically acceptable salt thereof.

In another embodiment the invention is concerned with the above methods wherein said administration is carried out by the oral, nasal, transdermal, pulmonal, or parenteral route.

In another embodiment the invention is concerned with the above methods wherein said disorder is selected from the group consisting of insulin resistance, diabetes type 1, diabetes type 2, metabolic syndrome X, impaired glucose tolerance, hyperglycemia, dyslipidemia, obesity, atheroschlerosis, hypertension, abnormalities of lipoprotein metabolism and any combination thereof.

In another embodiment the invention is concerned with the above methods wherein the therapeutically effective amount of the compound is from about 0.05 to about 2000 mg, preferably from about 0.1 to about 500 mg and even more preferable from about 1.0 to about 100 mg of said compound per day.

In another embodiment the invention is concerned with the above methods wherein a further antidiabetic, antiobesity, antihypertensive or appetite regulating drug is administered to the patient.

In another embodiment the invention is concerned with above methods wherein metformin is also administered to the patient.

The present invention also encompasses pharmaceutically acceptable salts of the present compounds. Such salts include pharmaceutically acceptable acid addition salts, pharmaceutically acceptable base addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium, zinc, calcium salts and the like. Examples of amines and organic amines include ammonium, methylamine, dimethylamine, trimethylamine, ethylamine, diethylamine, propylamine, butylamine, tetramethylamine, ethanolamine, diethanolamine, triethanolamine, meglumine, ethylenediamine, choline, N,N′-dibenzylethylenediamine, N-benzylphenylethylamine, N-methyl-D-glucamine, guanidine and the like. Examples of cationic amino acids include lysine, arginine, histidine and the like.

The pharmaceutically acceptable salts are prepared by reacting the compound of formula I with 1 to 4 equivalents of a base such as sodium hydroxide, sodium methoxide, sodium hydride, potassium t-butoxide, calcium hydroxide, magnesium hydroxide and the like, in solvents like ether, THF, methanol, t-butanol, dioxane, isopropanol, ethanol etc. Mixture of solvents may be used. Organic bases like lysine, arginine, diethanolamine, choline, guandine and their derivatives etc. may also be used. Alternatively, acid addition salts wherever applicable are prepared by treatment with acids such as hydrochloric acid, hydrobromic acid, nitric acid, sulfuric acid, phosphoric acid, p-toluenesulphonic acid, methanesulfonic acid, acetic acid, citric acid, maleic acid salicylic acid, hydroxynaphthoic acid, ascorbic acid, palmitic acid, succinic acid, benzoic acid, benzenesulfonic acid, tartaric acid and the like in solvents like ethyl acetate, ether, alcohols, acetone, THF, dioxane etc. Mixture of solvents may also be used.

The stereoisomers of the compounds forming part of this invention may be prepared by using reactants in their single enantiomeric form in the process wherever possible or by conducting the reaction in the presence of reagents or catalysts in their single enantiomer form or by resolving the mixture of stereoisomers by conventional methods. Some of the preferred methods include use of microbial resolution, enzymatic resolution, resolving the diastereomeric salts formed with chiral acids such as mandelic acid, camphorsulfonic acid, tartaric acid, lactic acid, and the like wherever applicable or chiral bases such as brucine, (R)— or (S)-phenylethylamine, cinchona alkaloids and their derivatives and the like. Commonly used methods are compiled by Jaques et al in “Enantiomers, Racemates and Resolution” (Wiley Inter-science, 1981). More specifically the compound of formula I may be converted to a 1:1 mixture of diastereomeric amides by treating with chiral amines, aminoacids, aminoalcohols derived from aminoacids; conventional reaction conditions may be employed to convert acid into an amide; the dia-stereomers may be separated either by fractional crystallization or chromatography and the stereoisomers of compound of formula I may be prepared by hydrolysing the pure diastereomeric amide.

Various polymorphs of compound of general formula I forming part of this invention may be prepared by crystallization of compound of formula I under different conditions. For example, using different solvents commonly used or their mixtures for recrystallization; crystallizations at different temperatures; various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffraction or such other techniques.

The invention also encompasses prodrugs of the present compounds, which on administration undergo chemical conversion by metabolic processes before becoming active pharmacological substances. In general, such prodrugs will be functional derivatives of the present compounds, which are readily convertible in vivo into the required compound of the formula I. Conventional procedures for the selection and preparation of suitable prodrug derivatives are described, for example, in “Design of Prodrugs”, ed. H. Bundgaard, Elsevier, 1985.

The invention also encompasses active metabolites of the present compounds.

The invention also relates to pharmaceutical compositions comprising, as an active ingredient, at least one compound of the formula I or any optical or geometric isomer or tautomeric form thereof including mixtures of these or a pharmaceutically acceptable salt thereof together with one or more pharmaceutically acceptable carriers or diluents.

Furthermore, the invention relates to the use of compounds of the general formula I or their tautomeric forms, their stereoisomers, their polymorphs, their pharmaceutically acceptable salts or pharmaceutically acceptable solvates thereof for the preparation of a pharmaceutical composition for the treatment and/or prevention of disorders where a decreased level of plasma FFA is desirable, such as the conditions mentioned above.

In another aspect, the present invention relates to a method of treating and/or preventing type 2 diabetes, insulin resistance, metabolic syndrome X, impaired glucose tolerance, dyslipidemia and abnormalities of lipoprotein metabolism.

In a still further aspect, the present invention relates to the use of one or more compounds of the general formula I, or pharmaceutically acceptable salts thereof, for the preparation of a pharmaceutical composition for the treatment and/or prevention of type 2 diabetes, insulin resistance, metabolic syndrome X, impaired glucose tolerance, dyslipidemia and abnormalities of lipoprotein metabolism.

In a still further aspect, the present compounds are useful for the delaying or prevention of the progression from impaired glucose tolerance to type 2 diabetes.

In a still further aspect, the present compounds are useful for the delaying or prevention of the progression from non-insulin requiring type 2 diabetes to insulin requiring type 2 diabetes.

In another aspect, the present compounds reduce triglyceride levels and are accordingly useful for the treatment and/or prevention of ailments and disorders such as diabetes and/or obesity.

In still another aspect, the compounds of general formula I are useful for the treatment of hyperglycemia, elevated HbA,, level, hyperinsulinemia, type 1.5 diabetes, latent autoimmune diabetes in adults, maturity onset diabetes, beta-cell apoptosis, hemochromatosis induced diabetes, impaired glucose tolerance, impaired fasting glucose, metabolic syndrome X, insulin resistance, impaired lipid tolerance, cystic fibrosis related diabetes, polycystic ovarian syndrome, and gestational diabetes.

In still another aspect, the compounds of general formula I are useful for the treatment of obesity, dyslipidemia, diabetic dyslipidemia, hyperlipidemia, hypertriglyceridemia, hyperlipoproteinemia, hypercholesterolemia, hypertension, essential hypertension, acute hypertensive emergency, arteriosclerosis, atherosclerosis, restenosis, intermittent claudication (atherosclerosis oblitterens), cardiovascular disease, cardiomyopathy, cardiac hypertrophy, left ventricular hypertrophy, coronary artery disease, early coronary artery disease, heart insufficiency, exercise tolerance, chronic heart failure, mild chronic heart failure, arrhythmia, cardiac dysrythmia, syncopy, heart attack, myocardial infarction, Q-wave myocardial infarction, stroke, acute coronary syndrome, angina pectoris, unstable angina, cardiac bypass reocclusion, diastolic dysfunction, systolic dysfunction, non-Q-wave cardiac necrosis, catabolic changes after surgery, acute pancreatitis, and irritable bowel syndrome

In still another aspect, the compounds of general formula I may be useful for the treatment of diabetic retinopathy, background retinopathy, preproliferative retinopathy, proliferative retinopathy, macular edema, cataracts, nephropathy, nephrotic syndrome, diabetic nephropathy, microalbuminuria, macroalbuminuria, neuropathy, diabetic neuropathy, distal symmetrical sensorimotor polyneuropathy, and diabetic autonomic neuropathy.

In still another aspect, the compounds of general formula I are useful for increasing the number of beta-cells in a patient, increasing the size of beta-cells in a patient or stimulating beta-cell proliferation, modulating beta-cell function and insulin secretion in a patient in need thereof, which method comprises administration of an effective amount of a compound of formula I to a patient in need thereof.

The compounds of the invention are also believed to be useful for reducing body weight in a patient in need thereof.

The compounds of the invention are also believed to be useful for weight neutral treatment of above mentioned diseases.

The compounds of the invention are also believed to be useful for redistributing fat in a patient in need thereof.

The compounds of the invention are also believed to be useful for redistributing central fat in a patient in need thereof.

The compounds of the invention are also believed to be useful for reducing or preventing central obesity.

The compounds of the invention are also believed to be useful for reducing postprandial serum lipid excursions.

The compounds of the invention are also believed to be useful for the treatment of fatty acid oxidation disorders such as MCAD.

In still another aspect, the compounds of general formula I are believed to be useful for the treatment of a disease, condition or disorder wherein cholesterol is a precursor. Such diseases, conditions or disorders may relate to testosterone, e.g. male contraception, excessive testosterone levels, PCOS and prostate cancer. They may also relate to cortisol or corticotropin, e.g. Cushing disease.

The compounds of the invention are also believed to be useful for the treatment of cancer.

Thus, the compounds of the general formula I may be useful for the treatment of insulinoma (pancreatic islet cell tumors), e.g. malignant insulinomas and multiple insulinomas, adipose cell carcinomas, e.g. lipocarconoma.

The compounds of the invention are also believed to be useful for the treatment of phaechromocytoma and other diseases with increased catecholamine incretion.

The compounds of the invention are also believed to be useful for the treatment of prostate cancer, e.g. adenocarcinoma.

In still another aspect, the compounds of general formula I may be useful for the treatment of hepatic steatosis.

In still another aspect, the compounds of general formula I may be useful for the treatment of cirrhosis.

In still another aspect, the compounds of general formula I may be useful for the treatment of AIDS or an AIDS related diseases, condition or disorders

In still another aspect, the compounds of general formula I may be useful for the treatment of lipodystrophy

In still another aspect, the compounds of general formula I may be useful for the treatment of lactic acidosis.

In yet another aspect, the compounds of the present invention are expected to be useful for the treatment of CNS diseases, conditions or disorders.

Thus, the compound of the present invention may be used for the treatment of Parkinsons disease, Alzheimers disease, ADHD (Attention Deficit Hyperactivity Disorder), feeding disorders such as bulimia and anorexia, depression, anxiety, cognitive memory disorders, age related cognitive decline, mild cognitive impairment and schizophrenia.

In yet another aspect, the compounds of the present invention may be useful for the treatment of inflammatory disorders, e.g. rheumatoid arthritis, psoriasis, systemic inflammatory response syndrome, sepsis and the like.

The present compounds may also be administered in combination with one or more further pharmacologically active substances eg., selected from antiobesity agents, antidiabetics, antihypertensive agents, agents for the treatment and/or prevention of complications resulting from or associated with diabetes and agents for the treatment and/or prevention of complications and disorders resulting from or associated with obesity.

Thus, in a further aspect of the invention the present compounds may be administered in combination with one or more antiobesity agents or appetite regulating agents.

Such agents may be selected from the group consisting of CART (cocaine amphetamine regulated transcript) agonists, NPY (neuropeptide Y) antagonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, β3 agonists, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, RXR (retinoid X receptor) modulators or TR β agonists.

In one embodiment of the invention the antiobesity agent is leptin.

In another embodiment the antiobesity agent is dexamphetamine or amphetamine.

In another embodiment the antiobesity agent is fenfluramine or dexfenfluramine.

In still another embodiment the antiobesity agent is sibutramine.

In a further embodiment the antiobesity agent is orlistat.

In another embodiment the antiobesity agent is mazindol or phentermine.

Suitable antidiabetics comprise insulin, exendin-4, GLP-1 (glucagon like peptide-1) and derivatives thereof such as those disclosed in WO 98/08871 to Novo Nordisk A/S, which is incorporated herein by reference as well as orally active hypoglycaemic agents.

The orally active hypoglycaemic agents preferably comprise sulphonylureas, biguanides, meglitinides, glucosidase inhibitors, glucagon antagonists such as those disclosed in WO 99/01423 to Novo Nordisk A/S and Agouron Pharmaceuticals, Inc., GLP-1 agonists, potassium channel openers such as those disclosed in WO 97/26265 and WO 99/03861 to Novo Nordisk A/S which are incorporated herein by reference, DPP-IV (dipeptidyl peptidase-IV) inhibitors, inhibitors of hepatic enzymes involved in stimulation of gluconeogenesis and/or glycogenolysis, glucose uptake modulators, compounds modifying the lipid metabolism such as antihyperlipidemic agents and antilipidemic agents as HMG CoA inhibitors (statins), compounds lowering food intake, RXR agonists and agents acting on the ATP-dependent potassium channel of the β-cells.

In one embodiment of the invention the present compounds are administered in combination with insulin.

In a further embodiment the present compounds are administered in combination with a sulphonylurea eg. tolbutamide, glibenclamide, glipizide or glicazide.

In another embodiment the present compounds are administered in combination with a biguanide eg. metformin.

In yet another embodiment the present compounds are administered in combination with a meglitinide eg. repaglinide or senaglinide.

In a further embodiment the present compounds are administered in combination with an α-glucosidase inhibitor eg. miglitol or acarbose.

In another embodiment the present compounds are administered in combination with an agent acting on the ATP-dependent potassium channel of the β-cells eg. tolbutamide, glibenclamide, glipizide, glicazide or repaglinide.

Furthermore, the present compounds may be administered in combination with nateglinide.

In still another embodiment the present compounds are administered in combination with an antihyperlipidemic agent or antilipidemic agent eg. cholestyramine, colestipol, clofibrate, gemfibrozil, lovastatin, pravastatin, simvastatin, probucol or dextrothyroxine.

In a further embodiment the present compounds are administered in combination with more than one of the above-mentioned compounds eg. in combination with a sulphonylurea and metformin, a sulphonylurea and acarbose, repaglinide and metformin, insulin and a sulphonylurea, insulin and metformin, insulin, insulin and lovastatin, etc.

Furthermore, the present compounds may be administered in combination with one or more antihypertensive agents. Examples of antihypertensive agents are -blockers such as alprenolol, atenolol, timolol, pindolol, propranolol and metoprolol, ACE (angiotensin converting enzyme) inhibitors such as benazepril, captopril, alatriopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin. Further reference can be made to Remington: The Science and Practice of Pharmacy, 19^{th} Edition, Gennaro, Ed., Mack Publishing Co., Easton, Pa., 1995.

It should be understood that any suitable combination of the compounds according to the invention with one or more of the above-mentioned compounds and optionally one or more further pharmacologically active substances are considered to be within the scope of the present invention.

The present invention also relates to processes according to reaction schemes P₁ and P₂ for the preparation of the above said novel compounds, their derivatives, their analogs, their tautomeric forms, their stereoisomers, their polymorphs, their pharmaceutically acceptable salts or pharmaceutically acceptable solvates.

The compounds of the invention may be administered alone or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19^{th} Edition, Gennaro, Ed., Mack Publishing Co., Easton, Pa., 1995. The compositions may appear in conventional forms, for example capsules, tablets, aerosols, solutions, suspensions or topical applications.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings such as enteric coatings or they can be formulated so as to provide controlled release of the active ingredient such as sustained or prolonged release according to methods well-known in the art.

Liquid dosage forms for oral administration include solutions, emulsions, suspensions, syrups and elixirs.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, cremes, gels, inhalants, dermal patches, implants etc.

The therapeutic dose of the compound will depend upon the frequency and mode of administration, the sex, age, weight and general condition of the subject treated, the nature and severity of the condition treated and any concomitant diseases to be treated and other factors evident to those skilled in the art. The formulations may conveniently be presented in unit dosage form by methods known to those skilled in the art. In one embodiment the composition in unit dosage form, comprises from about 0.05 to about 2000 mg, preferably from about 0.1 to about 500 mg of the compound of formula I pharmaceutically acceptable salt thereof.

In a still further embodiment the pharmaceutical composition is for oral, nasal, transdermal, pulmonal, or parenteral administration.

For parenteral routes, such as intravenous, intrathecal, intramuscular and similar administration, typically doses are in the order of about half the dose employed for oral administration.

The compounds of this invention are generally utilized as the free substance or as a pharmaceutically acceptable salt thereof. One example is an acid addition salt of a compound having the utility of a free base. When a compound of the invention contains a free base such salts are prepared in a conventional manner by treating a solution or suspension of a free base of the compound with a chemical equivalent of a pharmaceutically acceptable acid, for example, inorganic and organic acids. Representative examples are mentioned above. Physiologically acceptable salts of a compound with a hydroxy group include the anion of said compound in combination with a suitable cation such as sodium or ammonium ion.

For parenteral administration, solutions of the present compounds in sterile aqueous solution, aqueous propylene glycol or sesame or peanut oil may be employed. Such aqueous solutions should be suitable buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of suitable carriers are water, salt solutions, alcohols, polyethylene glycols, polyhydroxyethoxylated castor oil, peanut oil, olive oil, gelatine, lactose, terra alba, sucrose, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent may include any sustained release material known in the art, such as glyceryl monostearate or glyceryl distearate, alone or mixed with a wax. The formulations may also include wetting agents, emulsifying and suspending agents, preserving agents, sweetening agents or flavouring agents.

The pharmaceutical compositions formed by combining the compounds of the invention and the pharmaceutically acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and which may include a suitable excipient. These formulations may be in the form of powder or granules, as a solution or suspension in an aqueous or non-aqueous liquid, or as an oil-in-water or water-in-oil liquid emulsion.

If a solid carrier is used for oral administration, the preparation may be tabletted, placed in a hard gelatine capsule in powder or pellet form or it can be in the form of a troche or lozenge. The amount of solid carrier will vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

A typical tablet which may be prepared by conventional tabletting techniques may contain:

| | |
|---|---|
| Core: | |
| Active compound (as free compound or salt thereof) | 5 mg |
| Colloidal silicon dioxide (Aerosil) | 1.5 mg |
| Cellulose, microcryst. (Avicel) | 70 mg |
| Modified cellulose gum (Ac-Di-Sol) | 7.5 mg |
| Magnesium stearate | q.s. |
| Coating: | |
| HPMC approx. | 9 mg |
| *Mywacett 9-40 T approx. | 0.9 mg |

*Acylated monoglyceride used as plasticizer for film coating.

The compounds of the invention may be administered to a patient which is a mammal, especially a human in need thereof. Such mammals include also animals, both domestic animals, e.g. household pets, and non-domestic animals such as wildlife.

In a further aspect of the invention the present compounds may be administered in combination with further pharmacologically active substances e.g. an antidiabetic or other pharmacologically active material, including other compounds for the treatment and/or prevention of insulin resistance and diseases, wherein insulin resistance is the pathophysiological mechanism.

Furthermore, the compounds according to the invention may be administered in combination with antiobesity agents or appetite regulating agents.

### EXAMPLES

Materials

Methyl-phenyl-carbamic acid 4-hydroxymethyl-phenyl ester and methyl-phenyl-carbamic acid 4-(2-hydroxy-ethyl)-phenyl ester were prepared as described in PCT/DKO2/00852.

General Procedure 1.

A solution of an alcohol (0.4 mmol), a phenol/thiophenol (0.4 mmol), diisopropylethylamin (0.44 mmol) and tributylphosphine (0.52 mmol) in THF (5 mL) was stirred under nitrogen at rt. 1,1′-(Azodicarbonyl)dipiperidine (ADDP, 0.52 mmol) dissolved in THF (5 mL) was added and the reaction mixture was stirred at rt for 16 h. The reaction mixture was filtered and the filtrate was evaporated to dryness, which gave a crude which was either purified by flash chromatography (Quad flash 25, EtOAc-heptane) or redissolved in MeCN and purified by preparative HPLC (Gilson).

### Example 1

### General Procedure 1

### Methyl-phenyl-carbamic acid 4-[2-(pyridin-4-yloxy)-ethyl]-phenyl ester

The title compound (99%) was prepared as an oil using methyl-phenyl-carbamic acid 4-(2-hydroxy-ethyl)-phenyl ester and 4-hydroxypyridine.

HPLC-MS: m/z=349.2 (M+1); Rₜ=2.64 min.

### Example 2

### General Procedure 1

### Methyl-phenyl-carbamic acid 4-(1H-imidazol-2-ylsulfanylmethyl)-phenyl ester

The title compound (74%) was prepared as colorless crystals using methyl-phenyl-carbamic acid 4-hydroxymethyl-phenyl ester and 2-mercaptoimidazole. ¹H NMR (400 MHz; CDCl₃): δ 3.40 (br s, 3H), 4.10 (s, 2H), 6.94-7.13 (m, 6H),7.23-7.42 (m, 5H) 1H); HPLC-MS : m/z=340.0 (M+1); Rₜ=2.67 min.

### Example 3

### General Procedure 1

### Methyl-phenyl-carbamic acid 4-[2-(1H-imidazol-2-ylsulfanyl)-ethyl]-phenyl ester

The title compound (78%) was prepared as an oil using methyl-phenyl-carbamic acid 4-(2-hydroxy-ethyl)-phenyl ester and 2-mercaptoimidazole. ¹H NMR (400 MHz; CDCl₃): δ 2.83 (t, 2H), 3.11 (t, 2H), 3.40 (br s, 3H), 6.95-7.08 (m, 6H),7.20-7.39 (m, 5H); HPLC-MS: m/z=354.1 (M+1); Rₜ=2.77 min.

### Example 4

### Methyl-phenyl-carbamic acid 4-(6-methyl-pyridin-2-ylmethyl)-phenyl ester

To a stirred solution of (4-bromophenoxy)-tert-butylsilane (10 mmol) in THF (50 mL) was added dropwise 1.7 M solution in pentane t-BuLi (10 mmol) over a 15-min period at −78° C. The mixture was stirred at −78° C. for 2 min before 2-methyl-6-formylpyridine (10 mmol) dissolved in THF (5 mL) was added. The mixture was allowed to warm to room temperature and quenched with water. The pH of the aqueous phase was adjusted to 7 and extracted with CH₂Cl₂. The organic phases were dried and evaporated to give a crude which was dissolved in CH₂Cl₂ (40 mL) and stirred with triethylsilane (10 mL) and TFA (10 mL) for 16 h at 50° C. Evaporation and purification by flash chromatography (Quad flash 25, DCM: EtoAc 1:1 →0:1) gave 27% of 4-(6-Methyl-pyridin-2-ylmethyl)-phenol as yellow crystals.

4-(6-Methyl-pyridin-2-ylmethyl)-phenol (1 mmol)dissolved in DCM (10 mL) was added N-methyl-N-phenylcarbamoyl chloride (1.5 mmol) and DABCO (1.2 mmol) at room temperature. The reaction mixture was stirred for 16 hours at rt, filtered and evaporated to give a crude which was purified by FC (Quad flash 12 EtOAc-Heptane 1:1). This gave title compound (89%) as an oil. ¹H NMR (400 MHz; CDCl₃): δ 2.56 (s, 3H), 3.41 (br s, 3H), 4.11 (s, 2H), 6.82 (d, 1H), 6.96. (d, 1H), 7.05 (d, 2H), 7.20-7.45 (m, 8H); HPLC-MS : m/z=333.1 (M+1); Rₜ=3.08 min.

### Example 5

### Methyl-phenyl-carbamic acid 4-(4-methyl-pyridin-2-ylmethyl)-phenyl ester

### Example 6

### Methyl-phenyl-carbamic acid 4-(3-methyl-pyridin-2-ylmethyl)-phenyl ester

### Example 7

### Methyl-phenyl-carbamic acid 4-[2-(6-methyl-pyridin-2-yl)-ethyl]-phenyl ester

### Example 8

### Methyl-phenyl-carbamic acid 4-[2-(5-methyl-pyridin-2-yl)-ethyl]-phenyl ester

### Example 9

### Methyl-phenyl-carbamic acid 4-[2-(4-methyl-pyridin-2-yl)-ethyl]-phenyl ester

### Example 10

### Methyl-phenyl-carbamic acid 4-[2-(3-methyl-pyridin-2-yl)-ethyl]-phenyl ester

Pharmacological Methods

Compounds of formula I may be evaluated in vitro for their efficacy and potency to inhibit HSL, and such evaluation may be performed as described below.

Assays

Hormone-Ssensitive Lipase (HSL)

Materials. The Hormone-sensitive lipase was provided by Dr. Cecilia Holm, from Lund University Sweden or produced and purified by Novo Nordisk (NN) using the reagents and protocols used by Dr. Holm. The substrates used are: ³H-labeled triolein (TO) from Amersham, Buckinghamshire, U.K. cat No. TRA191; 5-20 Ci/mmol dissolved in toluene, triolein (Sigma, Cat. No. T-1740), fluorochrome-labeled triacylglyceride (cis-octadec-9-enoic acid 2-[12-(7-nitrobenzo[1,2,5]oxadiazol-4-ylamino)dodecanoyloxy]-1-cis-octadec-9-enoyloxymethyl-ethyl ester) prepared by Novo Nordisk (NN) by conventional methods, and 1,3-(di[³H]-stearin), 2-(PEG-Biotin)glycerol prepared in collaboration with Amersham Pharmacia Biotech, UK and described in WO 01/073442. Phosphatidyl choline (PC) and phosphatidyl inositol (PI) are from Sigma (St Luis Mo. cat. Nos. P-3556 and P-5954 respectively). All other reagents are of commercial grade and obtained from various commercial sources.

Methods.

3190.1: Assay for determination of percent inhibition of hormone sensitive lipase by compound at 10 μM sample concentration.

A lipid emulsion with fluorochrome-labeled triacylglyceride and phospholipid is used as substrate with a standard concentration of highly purified HSL (12 μg/mL initial concentration corresponding to 600 ng/mL final concentration). BSA is added as product acceptor. The transfer of the fluorochrome from the lipid phase to the water (BSA) phase changes the fluorescent properties of the fluorochrome. The changes can be monitored on a fluorimeter with an excitation wavelength of 450 nm and an emission wavelength of 545 nm.

Compound and HSL (20 μL compound, 10 μL enzyme and 70 μL PED-BSA buffer) is pre-incubated for 30 min at 25° C. before addition of substrate (100 μL). Amount of formed product is measured after 120 min incubation at 37° C.

Results are given as percent activity relative to a non-inhibited sample (no compound).

3190.2: Assay for determination of IC₅₀ value for the inhibition of hormone sensitive lipase by compound. Standard concentrations of compound are 100 μM and 5-fold dilutions (initial concentration corresponding to 10 μM final concentration and 5-fold).

A lipid emulsion with fluorochrome-labeled triacylglyceride and phospholipid is used as substrate with a standard concentration of highly purified HSL (12pg/mL initial concentration corresponding to 600 ng/mL final concentration). BSA is added as product acceptor. The transfer of the fluorochrome from the lipid phase to the water (BSA) phase changes the fluorescent properties of the fluorochrome. The changes can be monitored on a fluorimeter with an excitation wavelength of 450 nm and an emission wavelength of 545 nm.

Compound and HSL (20 μL compound, 10 μL enzyme and 79 μL PED-BSA buffer) is pre-incubated for 30 min at 25° C. before addition of substrate (100 μL). Amount of formed product is measured after 120 min incubation at 37° C.

Results are given as IC₅₀ values after 4PL fit of obtained activity data.

Results

With these methods the compounds of the examples are found to be inhibitors of HSL:

TABLE 1
Inhibition of HSL by compounds of the examples according to above
assay 3190.1 (% activity relative to non-inhibited sample).

| | | 3190.1 |
|---|---|---|
| | | HSL_FL |
| Example | | % ACTIVITY |
| No | Compound | (%) |
| 1 | Methyl-phenyl-carbamic acid 4-[2- | 1 |
| | (pyridin-4-yloxy)-ethyl]-phenyl | |
| | ester | |
| 2 | Methyl-phenyl-carbamic acid 4-(1H- | 1 |
| | imidazol-2-ylsulfanylmethyl)-phenyl | |
| | ester | |
| 3 | Methyl-phenyl-carbamic acid 4-[2-(1H- | 1 |
| | imidazol-2-ylsulfanyl)-ethyl]-phenyl | |
| | ester | |
| 4 | Methyl-phenyl-carbamic acid 4-(6-methyl- | 5 |
| | pyridin-2-ylmethyl)-phenyl ester | |

1. A compound of formula (I): 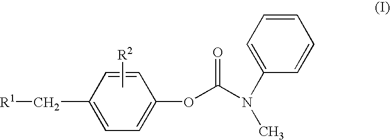
wherein
R¹ is selected from hydroxy, sulfanyl, sulfo, C₁₋₆alkyl, C₁₋₆-alkoxy, C₂₋₆-alkenyl, aryl, heteroaryl, C₃₋₈-heterocyclyl and C₃₋₁₀-cycloalkyl, wherein each of hydroxy, sulfanyl, sulfo, C₁₋₆alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, C₁₋₆alkyl, C₁₋₆alkoxy, C₂₋₆-alkenyl, aryl, heteroaryl, C₃₋₈-heterocyclyl, and C₃₋₁₀-cycloalkyl, wherein each of hydroxy, sulfanyl, sulfo, C₁₋₆-alkyl, C₁₋₆alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;
R² is selected from hydrogen, hydroxy, sulfanyl, amino, halogen, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₁₋₆alkoxy, C₂₋₆-alkenyl, aryl, heteroaryl, C₃₋₈m-heterocyclyl and C₃₋₁₀-cycloalkyl may optionally be substituted with one or more substituents independently selected from hydroxy, sulfanyl, oxo, halogen, amino, sulfo, C₁₋₆alkyl, C₁₋₆-alkoxy, C₂₋₆-alkenyl, aryl, heteroaryl, C₃₋₈-heterocyclyl, and C₃₋₁₀-cycloalkyl, wherein each of hydroxy, sulfanyl, amino, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;
with the proviso that said compound is not
Methyl-phenyl-carbamic acid 4-(3-chloro-5-trifluoromethyl)-pyridine-2-ylmethyl)-phenyl ester,
Methyl-phenyl-carbamic acid 4-(1,3,5-trimethyl-1H-pyrazol-4-ylmethyl)-phenyl ester,
Methyl-phenyl-carbamic acid 4-(2-cyano-ethyl)-phenyl ester,
Methyl-phenyl-carbamic acid 4-pentyl-phenyl ester,
Methyl-phenyl-carbamic acid 4-(2-methoxy-ethyl)-phenyl ester,
Methyl-phenyl-carbamic acid 4-[2-(4-chloro-phenyl)-ethyl]-phenyl ester,
Methyl-phenyl-carbamic acid 4-(4-hydroxy-benzyl)-phenyl ester,{2-[4-(Methyl-phenyl-carbamoyloxy)-phenyl]-ethyl}-carbamic acid tert-butyl ester,
Methyl-phenyl-carbamic acid 4-(2-amino-ethyl)phenyl ester,
Methyl-phenyl-carbamic acid 4-[2-(toluene-4-sulfonylamino)-ethyl]-phenyl ester,
Methyl-phenyl-carbamic acid 4-[2-(5-dimethylamino-naphthalene-1-sulfonylamino)-ethyl]-phenyl ester,
Methyl-phenyl-carbamic acid 4-[2-(3,4-difluoro-benzenesulfonylamino)-ethyl]-phenyl ester,
2-{2-[4-(Methyl-phenyl-carbamoyloxy)-phenyl]-ethylsulfamoyl}-benzoic acid methyl ester,
Methyl-phenyl-carbamic acid 4-[2-(2,5-dichloro-thiophene-3-sulfonylamino)-ethyl]-phenyl ester,
Methyl-phenyl-carbamic acid 4-[2-(5-pyridin-2-yl-thiophene-2-sulfonylamino)-ethyl]-phenyl ester,
Methyl-phenyl-carbamic acid 4-[2-(1-methyl-1H-imidazole-4-sulfonylamino)-ethyl]-phenyl ester,
Methyl-phenyl-carbamic acid 4-[2-(5-chloro-1,3-dimethyl-1H-pyrazole-4-sulfonylamino)-ethyl]-phenyl,
Methyl-phenyl-carbamic acid 4-[2-(4-nitro-benzenesulfonylamino)-ethyl]-phenyl ester,
Methyl-phenyl-carbamic acid 4-[2-(6-chloro-imidazo[2,1-b]thiazole-5-sulfonylamino)-ethyl]-phenyl ester,
Methyl-phenyl-carbamic acid 4-[2-(2-trifluoromethoxy-benzenesulfonylamino)-ethyl]-phenyl ester,
Methyl-phenyl-carbamic acid 4-(2-dimethylaminomethanesulfonylamino-ethyl)-phenyl ester,
Methyl-phenyl-carbamic acid 4-(2-methanesulfonylamino-ethyl)-phenyl ester,
Methyl-phenyl-carbamic acid 4-[2-(6-morpholin-4-yl-pyridine-3-sulfonylamino)-ethyl]-phenyl ester,
Methyl-phenyl-carbamic acid 4-[2-(6-phenoxy-pyridine-3-sulfonylamino)-ethyl]-phenyl ester,
Methyl-phenyl-carbamic acid 4-{2-[4-(4-methyl-piperazin-1-yl)-benzenesulfonylamino]-ethyl}-phenyl ester,
Methyl-phenyl-carbamic acid 4-[2-(4-dimethylamino-benzenesulfonylamino)-ethyl]-phenyl ester,
Methyl-phenyl-carbamic acid 4-{2-[4-(2-pyrrolidin-1-yl-ethoxy)-benzenesulfonylamino]-ethyl}-phenyl ester,
Methyl-phenyl-carbamic acid 4-pyridin-2-ylmethyl-phenyl ester,
Methyl-phenyl-carbamic acid 4-pyridin-3-ylmethyl-phenyl ester,
Methyl-phenyl-carbamic acid 4-(4-trifluoromethyl-benzyl)-phenyl ester,
Methyl-phenyl-carbamic acid 4-thiophen-3-ylmethyl-phenyl ester,
Methyl-phenyl-carbamic acid 4-thiophen-2-ylmethyl-phenyl ester,
Methyl-phenyl-carbamic acid 4-[2-(4-amino-benzenesulfonylamino)-ethyl]-phenyl ester,
Methyl-phenyl-carbamic acid 4-{2-[(pyridine-3-carbonyl)-amino]-ethyl}-phenyl ester,
Methyl-phenyl-carbamic acid 4-[2-(2-dimethylamino-acetylamino)-ethyl]-phenyl ester,
Methyl-phenyl-carbamic acid 2-(toluene4-sulfonyl)-1,2,3,4-tetrahydro-isoquinolin-7-yl ester,
Methyl-phenyl-carbamic acid 4-[4-(2-pyrrolidin-1-yl-ethoxy)-benzyl]-phenyl ester,
Methyl-phenyl-carbamic acid 4-{2-[(1-methyl-piperidine-4-carbonyl)-amino]-ethyl}-phenyl ester,
3,3-Dimethyl-4-{2-[4-(methyl-phenyl-carbamoyloxy)-phenyl]-ethylcarbamoyl}-butyric acid,
Methyl-phenyl-carbamic acid 4-{2-[4-(4-methyl-piperazin-1-yl)-benzoylamino]-ethyl}-phenyl ester,
Methyl-phenyl-carbamic acid 4-{2-[4-(4-methyl-piperazin-1-ylmethyl)-benzoylamino]-ethyl}-phenyl ester,
Methyl-phenyl-carbamic acid 4-[2-(4,4-dimethyl-2,6-dioxo-piperidin-1-yl)-ethyl]-phenyl ester,
3,3-Dimethyl-4-{2-[4-(methyl-phenyl-carbamoyloxy)-phenyl]-ethylcarbamoyl}-butyric acid ethyl ester,
Methyl-phenyl-carbamic acid 4-hydroxymethyl-phenyl ester,
Methyl-phenyl-carbamic acid 4-(2-hydroxy-ethyl)-phenyl ester,
Methyl-phenyl-carbamic acid 4-(4-dimethylamino-pyridin-2-ylmethyl)-phenyl ester,
Methyl-phenyl-carbamic acid 4-(4-imidazol-1-yl-phenoxymethyl)-phenyl ester,
Methyl-phenyl-carbamic acid 4-[4-(2-dimethylamino-ethyl)-phenoxymethyl]-phenyl ester,
Methyl-phenyl-carbamic acid 4-(pyrazol-1-yloxymethyl)-phenyl ester,
Methyl-phenyl-carbamic acid 4-(imidazol-1-yloxymethyl)-phenyl ester,
Methyl-phenyl-carbamic acid 4-(2-oxo-2H-pyridin-1-ylmethyl)-phenyl ester,
Methyl-phenyl-carbamic acid 4-[2-(pyridin-2-yloxy)-ethyl]-phenyl ester,
Methyl-phenyl-carbamic acid 4-[2-(4-imidazol-1-yl-phenoxy)-ethyl]-phenyl ester,
Methyl-phenyl-carbamic acid 4-{2-[4-(2-dimethylamino-ethyl)-phenoxy]-ethyl}-phenyl ester,
Methyl-phenyl-carbamic acid 4-[2-(pyrazol-1-yloxy)-ethyl]-phenyl ester,
Methyl-phenyl-carbamic acid 4-[2-(imidazol-1-yloxy)-ethyl]-phenyl ester,
Methyl-phenyl-carbamic acid 4-(5-methyl-pyridin-2-ylmethyl)-phenyl ester,
Methyl-phenyl-carbamic acid 4-(4-oxo-4H-pyridin-1-ylmethyl)-phenyl ester,
Methyl-phenyl-carbamic acid 4-[2-(pyridin-3-yloxy)-ethyl]-phenyl ester,
Methyl-phenyl-carbamic acid 4-(2-oxo-2H-pyridin-1-ylmethyl)-phenyl ester,
Methyl-phenyl-carbamic acid 4-(pyridin-3-yloxymethyl)-phenyl ester,
Methyl-phenyl-carbamic acid 4-[2-(2,5-dioxo-pyrrolidin-1-yl)-ethyl]-phenyl ester,
Methyl-phenyl-carbamic acid 4-[2-(1,3-dioxo-1,3-dihydro-pyrrolo[3,4-]pyridin-2-yl)-ethyl]-phenyl ester,
Methyl-phenyl-carbamic acid 4-(1-methyl-1H-imidazol-2-ylsulfanylmethyl)-phenyl ester,
Methyl-phenyl-carbamic acid 4-tetrazol-1-ylmethyl-phenyl ester,
Methyl-phenyl-carbamic acid 4-(2,5-dioxo-pyrrolidin-1-ylmethyl)-phenyl ester,
Methyl-phenyl-carbamic acid 4-[2-(2-thioxo-2H-pyridin-1-yl)-ethyl]-phenyl ester,
Methyl-phenyl-carbamic acid 4-(1,3-dioxo-1,3-dihydro-pyrrolo[3,4)pyrid in-2-ylmethyl)-phenyl ester,
Methyl-phenyl-carbamic acid 4-[1,2,4]triazol-1-ylmethyl-phenyl ester,
Methyl-phenyl-carbamic acid 4-(2-thioxo-2H-pyridin-1-ylmethyl)-phenyl ester,
Methyl-phenyl-carbamic acid 4-[2-(1-methyl-1H-imidazol-2-ylsulfanyl)-ethyl]-phenyl ester,ethyl-phenyl-carbamic acid 4-(2-tetrazol-1-yl-ethyl)-phenyl ester, Methyl-phenyl-carbamic acid 4-[2-(pyrimidin-2-yloxy)-ethyl]-phenyl ester,
Methyl-phenyl-carbamic acid 4-[2-(pyridin-4-ylsulfanyl)-ethyl]-phenyl ester,
Methyl-phenyl-carbamic acid 4-[2-(1-pyridin-3-yl-1H-imidazol-2-ylsulfanyl)-ethyl]-phenyl ester,
Methyl-phenyl-carbamic acid 4-[2-(1,3-dioxo-1,3-dihydro-isoindol-2-yl)-ethyl]-phenyl ester, and
Methyl-phenyl-carbamic acid 4-benzyl-phenyl ester;
as well as diastereomers, enantiomers or tautomeric forms thereof, mixtures of these, pharmaceutically acceptable salts thereof, pharmaceutically acceptable solvates thereof, or polymorphs.

2. A compound according to claim 1, of formula (Ia): 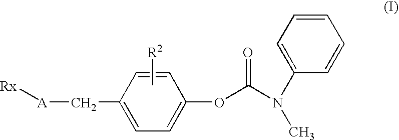
wherein
Rx is selected from aryl, heteroaryl, C₃₋₈-heterocyclyl and C₃₋₁₀-cycloalkyl, wherein each of aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, C₁₋₆-alkyl, C₁₋₆-alkoxy, C₂₋₆-alkenyl, aryl, heteroaryl, C₃₋₈-heterocyclyl, and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;
A is selected from —CH₂O—, —CH₂S—, —S—, —O—, —S(═O)₂—, —CH₂(S═O)₂, and —CH₂—, or is absent;
R² is selected from hydrogen, hydroxy, sulfanyl, amino, halogen, sulfo, C₁₋₆-alkyl, C₁₋₆-alkoxy, C₂₋₆-alkenyl, aryl, heteroaryl, C₃₋₈heterocyclyl and C₃₋₁₀-cycloalkyl, wherein each of hydroxy, sulfanyl, amino, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, C₁₋₆-alkyl, C₁₋₆-alkoxy, C₂₋₆-alkenyl, aryl, heteroaryl, C₃₋₈-heterocyclyl, and C₃₋₁₀-cycloalkyl, wherein each of hydroxy, sulfanyl, amino, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;
with the proviso that said compound is not
Methyl-phenyl-carbamic acid 4-(3-chloro-5-trifluoromethyl)-pyridine-2-ylmethyl)-phenyl ester,
Methyl-phenyl-carbamic acid 4-(1,3,5-trimethyl-1H-pyrazol-4-ylmethyl)-phenyl ester,
Methyl-phenyl-carbamic acid 4-[2-(1,3-dioxo-1,3-dihydro-pyrrolo[3,4-]pyridin-2-yl)-ethyl]-phenyl ester,
Methyl-phenyl-carbamic acid 4-(1,3-dioxo-1,3-dihydro-pyrrolo[3,4)pyridin-2-ylmethyl)-phenyl ester,
Methyl-phenyl-carbamic acid 4-[2-(1,3-dioxo-1,3-dihydro-isoindol-2-yl)-ethyl]-phenyl ester,
Methyl-phenyl-carbamic acid 4-[2-(4-chloro-phenyl)-ethyl]-phenyl ester,
Methyl-phenyl-carbamic acid 4-(4-hydroxy-benzyl)-phenyl ester,
Methyl-phenyl-carbamic acid 4-pyridin-2-ylmethyl-phenyl ester,
Methyl-phenyl-carbamic acid 4-pyridin-3-ylmethyl-phenyl ester,
Methyl-phenyl-carbamic acid 4-(4-trifluoromethyl-benzyl)-phenyl ester,
Methyl-phenyl-carbamic acid 4-thiophen-3-ylmethyl-phenyl ester,
Methyl-phenyl-carbamic acid 4-thiophen-2-ylmethyl-phenyl ester,
Methyl-phenyl-carbamic acid 4-[4-(2-pyrrolidin-1-yl-ethoxy)-benzyl]-phenyl ester,
Methyl-phenyl-carbamic acid 4-(4-dimethylamino-pyridin-2-ylmethyl)-phenyl ester,
Methyl-phenyl-carbamic acid 4-(4-imidazol-1-yl-phenoxymethyl)-phenyl ester,
Methyl-phenyl-carbamic acid 4-(pyrazol-1-yloxymethyl)-phenyl ester,
Methyl-phenyl-carbamic acid 4-(imidazol-1-yloxymethyl)-phenyl ester,
Methyl-phenyl-carbamic acid 4-[2-(pyridin-2-yloxy)-ethyl]-phenyl ester,
Methyl-phenyl-carbamic acid 4-[2-(4-imidazol-1-yl-phenoxy)-ethyl]-phenyl ester,
Methyl-phenyl-carbamic acid 4-[2-(pyrazol-1-yloxy)-ethyl]-phenyl ester,
Methyl-phenyl-carbamic acid 4-[2-(imidazol-1-yloxy)-ethyl]-phenyl ester,
Methyl-phenyl-carbamic acid 4-(5-methyl-pyridin-2-ylmethyl)-phenyl ester,
Methyl-phenyl-carbamic acid 4-(4-oxo-4H-pyridin-1-ylmethyl)-phenyl ester,
Methyl-phenyl-carbamic acid 4-[2-(pyridin-3-yloxy)-ethyl]-phenyl ester,
Methyl-phenyl-carbamic acid 4-(2-oxo-2H-pyridin-1-ylmethyl)-phenyl ester,
Methyl-phenyl-carbamic acid 4-(pyridin-3-yloxymethyl)-phenyl ester,
Methyl-phenyl-carbamic acid 4-[2-(2,5-dioxo-pyrrolidin-1-yl)-ethyl]-phenyl ester,
Methyl-phenyl-carbamic acid 4-(1-methyl-1H-imidazol-2-ylsulfanylmethyl)-phenyl ester,
Methyl-phenyl-carbamic acid 4-tetrazol-1-ylmethyl-phenyl ester,
Methyl-phenyl-carbamic acid 4-(2,5-dioxo-pyrrolidin-1-ylmethyl)-phenyl ester,
Methyl-phenyl-carbamic acid 4-[1,2,4]triazol-1-ylmethyl-phenyl ester,
Methyl-phenyl-carbamic acid 4-[2-(1-methyl-1H-imidazol-2-ylsulfanyl)-ethyl]-phenyl ester,
Methyl-phenyl-carbamic acid 4-(2-tetrazol-1-yl-ethyl)-phenyl ester,
Methyl-phenyl-carbamic acid 4-[2-(pyrimidin-2-yloxy)-ethyl]-phenyl ester,
Methyl-phenyl-carbamic acid 4-[2-(pyridin-4-ylsulfanyl)-ethyl]-phenyl ester,
Methyl-phenyl-carbamic acid 4-[2-(1-pyridin-3-yl-1H-imidazol-2-ylsulfanyl)-ethyl]-phenyl ester;
as well as diastereomers, enantiomers or tautomeric forms thereof, mixtures of these, pharmaceutically acceptable salts thereof, pharmaceutically acceptable solvates thereof, or polymorphs.

3. A compound according to claim 2, wherein
Rx is selected from aryl, heteroaryl, C₃₋₈-heterocyclyl, and C₃₋₁₀-cycloalkyl;
A is selected from —CH₂O—, —CH₂S—, —CH₂—,
R² is hydrogen;
with the proviso that said compound is not
Methyl-phenyl-carbamic acid 4-[2-(pyridin-2-yloxy)-ethyl]-phenyl ester,
Methyl-phenyl-carbamic acid 4-[2-(pyrazol-1-yloxy)-ethyl]-phenyl ester,
Methyl-phenyl-carbamic acid 4-[2-(imidazol-1-yloxy)-ethyl]-phenyl ester,
Methyl-phenyl-carbamic acid 4-[2-(pyridin-3-yloxy)-ethyl]-phenyl ester,
Methyl-phenyl-carbamic acid 4-(2-tetrazol-1-yl-ethyl)-phenyl ester,
Methyl-phenyl-carbamic acid 4-[2-(pyrimidin-2-yloxy)-ethyl]-phenyl ester, and
Methyl-phenyl-carbamic acid 4-[2-(pyridin-4-ylsulfanyl)-ethyl]-phenyl ester;
as well as diastereomers, enantiomers or tautomeric forms thereof, mixtures of these, pharmaceutically acceptable salts thereof, pharmaceutically acceptable solvates thereof, or polymorphs.

4. A compound according to claim 2, wherein
Rx is selected from aryl, heteroaryl, C₃₋₈-heterocyclyl, and C₃₋₁₀-cycloalkyl, wherein each of aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;
A is selected from —S—, —O—, —S(═O)₂—, —CH₂—,
R² is hydrogen;
with the proviso that said compound is not
Methyl-phenyl-carbamic acid 4-(2-oxo-2H-pyridin-1-ylmethyl)-phenyl ester,
Methyl-phenyl-carbamic acid 4-[2-(1,3-dioxo-1,3-dihydro-pyrrolo[3,4-]pyridin-2-yl)-ethyl]-phenyl ester,
Methyl-phenyl-carbamic acid 4-[2-(1,3-dioxo-1,3-dihydro-isoindol-2-yl)-ethyl]-phenyl ester,
Methyl-phenyl-carbamic acid 4-[2-(4,4-dimethyl-2,6-dioxo-piperidin-1-yl)-ethyl]-phenyl ester,
Methyl-phenyl-carbamic acid 4-[2-(4-chloro-phenyl)-ethyl]-phenyl ester,
Methyl-phenyl-carbamic acid 4-(4-imidazol-1-yl-phenoxymethyl)-phenyl ester,
Methyl-phenyl-carbamic acid 4-(pyrazol-1-yloxymethyl)-phenyl ester,
Methyl-phenyl-carbamic acid 4-(imidazol-1-yloxymethyl)-phenyl ester,
Methyl-phenyl-carbamic acid 4-(pyridin-3-yloxymethyl)-phenyl ester,
Methyl-phenyl-carbamic acid 4-[2-(2,5-dioxo-pyrrolidin-1-yl)-ethyl]-phenyl ester,
Methyl-phenyl-carbamic acid 4-(1-methyl-1H-imidazol-2-ylsulfanylmethyl)-phenyl ester, and
Methyl-phenyl-carbamic acid 4-(2-tetrazol-1-yl-ethyl)-phenyl ester;
as well as diastereomers, enantiomers or tautomeric forms thereof, mixtures of these, pharmaceutically acceptable salts thereof, pharmaceutically acceptable solvates thereof, or polymorphs.

5. A compound according to claim 1, wherein R² is hydrogen.

6. A compound according to claim 1, wherein R² is selected from hydrogen, hydroxy, sulfanyl, amino, halogen, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, C₁₋₆-alkyl, C₁₋₆alkoxy, C₂₋₆-alkenyl, perhalomethyl, and perhalomethoxy.

7. A compound according to claim 1, wherein R² is selected from the group consisting of fluor, chlor, amino, —COOH, C₁₋₆-alkyl, C₁₋₆-alkoxy, and C₁₋₆-alkyl which has been substituted with one or more halogens.

8. A compound according to claim 1, wherein R² is selected from the group consisting of

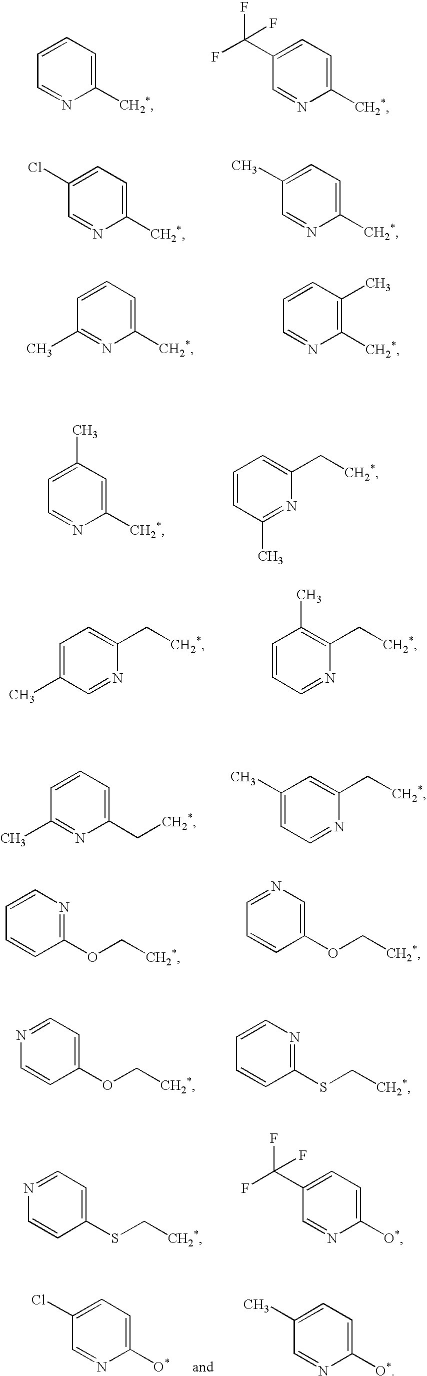

9. A compound according to claim 1, wherein R² is selected from the group consisting of

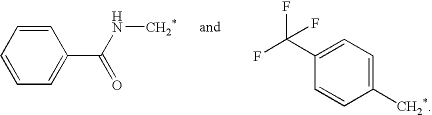

10. A compound according to claim 1, wherein R² is selected from the group consisting of

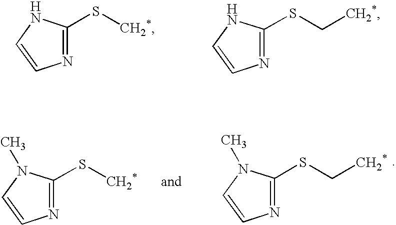

11. A compound according to claim 1, wherein R² is selected from the group consisting of

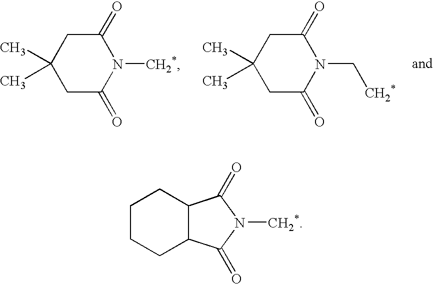

12. A compound according to claim 1, wherein R¹ is selected from the group consisting of

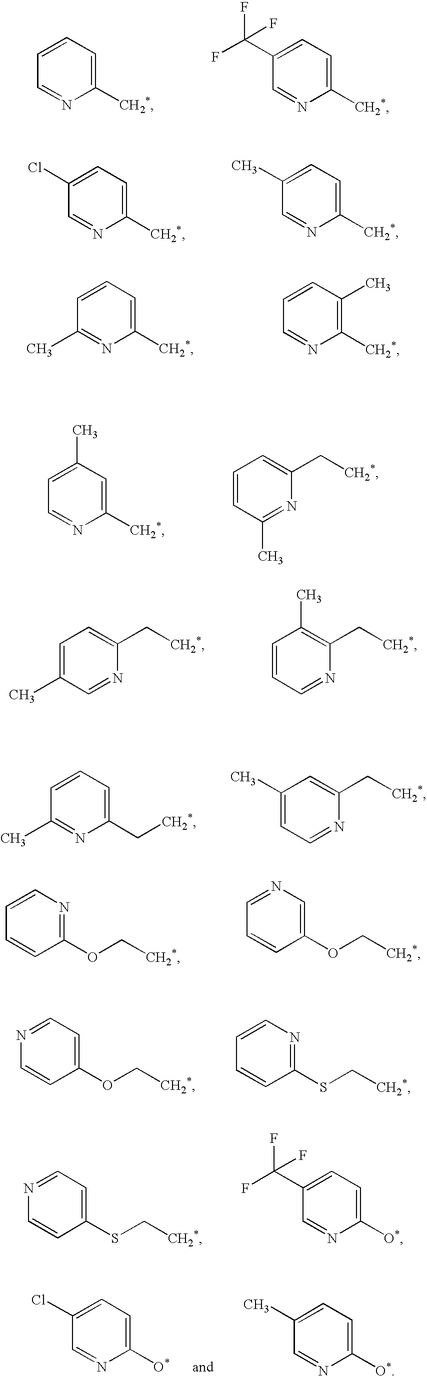

13. A compound according to claim 1, wherein R¹ is selected from the group consisting of

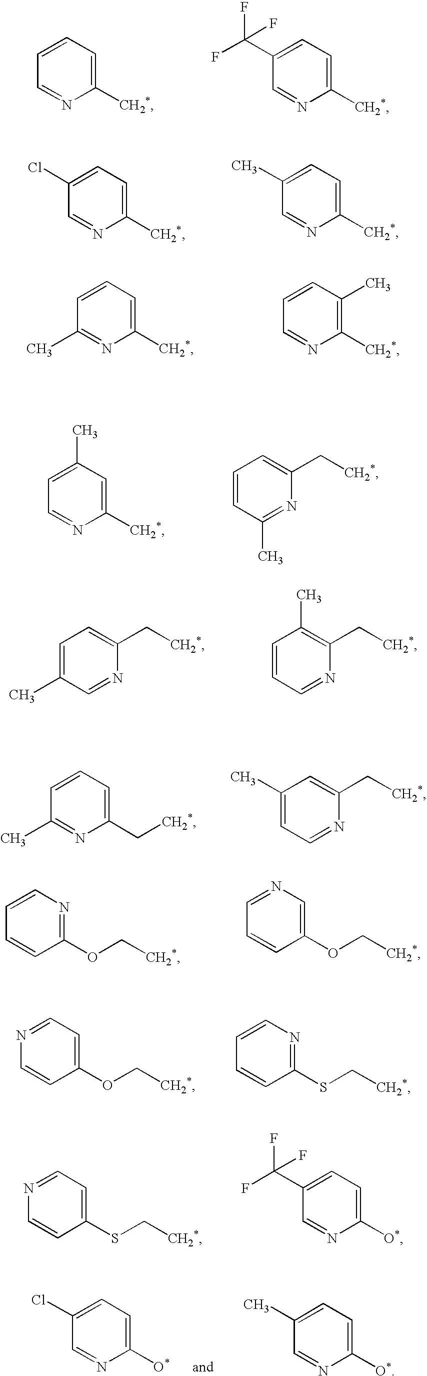

14. A compound according to claim 1, wherein R¹ is selected from the group consisting of

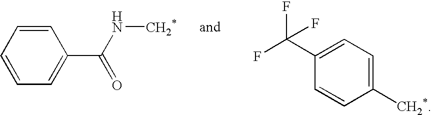

15. A compound according to claim 1, wherein R¹ is selected from the group consisting of

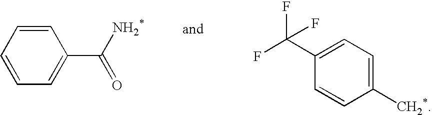

16. A compound according to claim 1, wherein R¹ is selected from the group consisting of

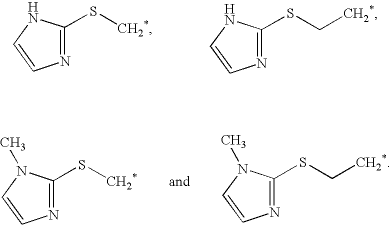

17. A compound according to claim 1, wherein R¹ is selected from the group consisting of

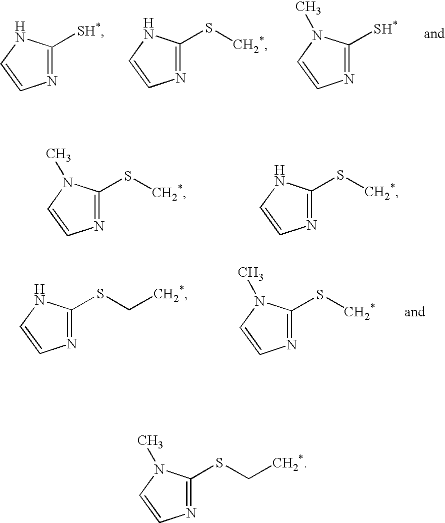

18. A compound according to claim 1, wherein R¹ is selected from the group consisting of

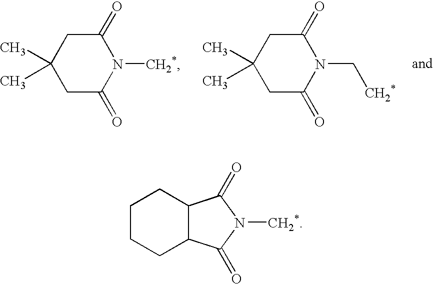

19. A compound according to claim 1, wherein R¹ is selected from the group consisting of

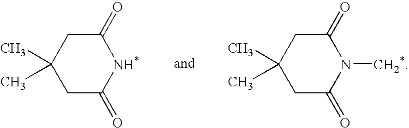

20. A compound according to claim 1, wherein R¹ contains an aryl or a hetteroaryl group.

21. A compound according to claim 1, wherein R² is hydrogen.

22. A compound according to claim 1, wherein the sum of the molar weights of the substituents R¹ and R² is in the range from about 60 g/mole to about 250 g/mole.

23. A compound according to claim 1, having one free —COOH group.

24. A compound according to claim 1, having one free amino group, or one monosubstituted amino group or one disubstituted amino group.

25. A compound according to claim 1, having one substituted or unsubstituted pyridine ring.

26. A compound according to claim 1, having one substituted or unsubstituted imidazole ring.

27. A compound according to claim 1, wherein the molar weight of said compound is less than 650 g/mole.

28. A compound according to claim 1, wherein the compound contains no ionisable group and wherein cLog P is in the range from 1.0 to 5.0.

29. A compound according to claim 1, wherein the compound contains no ionisable group and wherein cLog P is in the range from 3.0 to 5.0.

30. A compound according to claim 1, wherein the ACD LogD is in the range from 0.8 to 3.0.

31. A compound according to claim 1, wherein the number of H-bond donors is 0, 1, or 3.

32. A compound according to claim 1, wherein the number of H-bond donors is 0 or 1.

33. A compound according to claim 1, wherein the number of H-bond acceptors is in the range from 4 to 9.

34. A compound according to claim 1, wherein the number of H-bond acceptors is in the range from 4 to 7.

35. A compound according to claim 1, wherein the number of rotatable bonds of said compound is in the range from 4 to 14.

36. A compound according to claim 1, wherein the number of rotatable bonds of said compound is in the range from 6 to 10.

37. A compound according to claim 1, wherein the polar surface area (PSA) is in the range from 30 Å² to120 Å².

38. A compound according to claim 1, wherein the polar surface area (PSA) is in the range from 30 Å² to80 Å².

39. A compound according to claim 1, wherein the compound is selected from the group consisting of
Methyl-phenyl-carbamic acid 4-[2-(pyridin-4-yloxy)-ethyl]-phenyl ester,
Methyl-phenyl-carbamic acid 4-(1H-imidazol-2-ylsulfanylmethyl)-phenyl ester,
Methyl-phenyl-carbamic acid 4-[2-(1H-imidazol-2-ylsulfanyl)-ethyl]-phenyl ester,
Methyl-phenyl-carbamic acid 4-(6-methyl-pyridin-2-ylmethyl)-phenyl ester,
Methyl-phenyl-carbamic acid 4-(4-methyl-pyridin-2-ylmethyl)-phenyl ester,
Methyl-phenyl-carbamic acid 4-(3-methyl-pyridin-2-ylmethyl)-phenyl ester,
Methyl-phenyl-carbamic acid 4-[2-(6-methyl-pyridin-2-yl)-ethyl]-phenyl ester,
Methyl-phenyl-carbamic acid 4-[2-(5-methyl-pyridin-2-yl)-ethyl]-phenyl ester,
Methyl-phenyl-carbamic acid 4-[2-(4-methyl-pyridin-2-yl)-ethyl]-phenyl ester, and
Methyl-phenyl-carbamic acid 4-[2-(3-methyl-pyridin-2-yl)-ethyl]-phenyl ester.

40. A pharmaceutical composition comprising a compound according to claim 1, or a pharmaceutically acceptable salt thereof together with a pharmaceutically acceptable carrier or diluent.

41. The composition according to claim 40, wherein said composition is in unit dosage form, comprising from about 0.05 to about 2000 mg, from about 0.1 to about 500 mg, or from about 1.0 to about 100 mg of said compound according to any one of claims 1-39 or pharmaceutically acceptable salt thereof.

42. A pharmaceutical composition for use as a medicament for inhibiting the lipolytic activity of hormone-sensitive lipase against triacylglycerols, diacylglycerols, cholesterol acyl esters or steroid acyl esters, said composition comprising a compound according to claim 1, or a pharmaceutically acceptable salt thereof together with a pharmaceutically acceptable carrier or diluent.

43. A pharmaceutical composition according to claim 40, which is for oral administration.

44. A pharmaceutical composition according to claim 40, for nasal, transdermal, pulmonal, or parenteral administration.

45. A method of treating a disorder of a patient where modulation of the activity of hormone-sensitive lipase is desired, the method comprising administering to a subject in need thereof a therapeutically effective amount of a compound according to claim 1, or a pharmaceutically acceptable salt thereof.

46. A method of treating a disorder of a patient where lowering of the activity of hormone-sensitive lipase is desired, the method comprising administering to a subject in need thereof a therapeutically effective amount of a compound according to claim 1, or a pharmaceutically acceptable salt thereof.

47. The method according to claim 45, wherein said administration is carried out by the oral, nasal, transdermal, pulmonal, or parenteral route.

48. The method according to claim 45, wherein said disorder is selected from the group consisting of insulin resistance, diabetes type 1, diabetes type 2, metabolic syndrome X, impaired glucose tolerance, hyperglycemia, dyslipidemia, obesity, atheroschlerosis, hypertension, abnormalities of lipoprotein metabolism and any combination thereof.

49. The method according to claim 45, wherein the therapeutically effective amount of the compound is from about 0.05 to about 2000 mg, from about 0.1 to about 500 mg, or from about 1.0 to about 100 mg of said compound per day.

50. The method according to claim 45, wherein a further antidiabetic, antiobesity, antihypertensive or appetite regulating drug is administered to the patient.

51. The method according to claim 45, wherein metformin is also administered to the patient.

52. A process for the preparation of a compound according to claim 1, or its pharmaceutically acceptable salt, comprising reacting the appropriate alcohol with the appropriate carbamoylating reagent in a solvent according to the reaction scheme P₁ 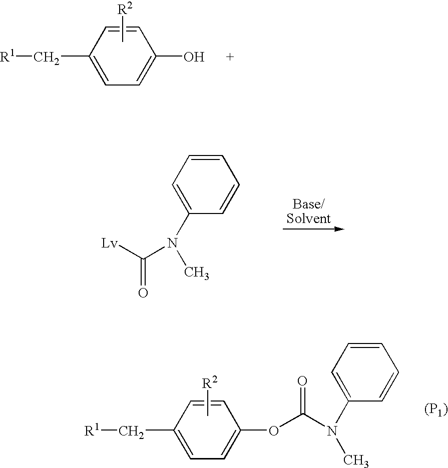
and isolating the disubstituted carbamate product.

53. The process according to claim 52, wherein said carbamoylating reagent 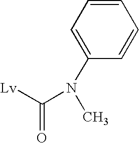
is selected from the group consisting of

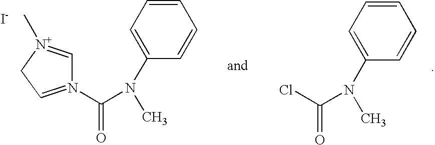

54. The process according to claim 52, wherein said solvent is selected from the group consisting of tetrahydrofurane, dimethylformamide and N-methylpyrolidone.

55. The process according to claim 52, wherein said base is selected from the group consisting of triethylamine, N,N-diisopropyl-N-ethylamine and DABCO.

56. A process for the preparation of a compound according to claim 1, said process comprising the treatment of the appropriate amine with the appropriate acylating reagent in a solvent and in the presence of a base according to the reaction scheme P₂ 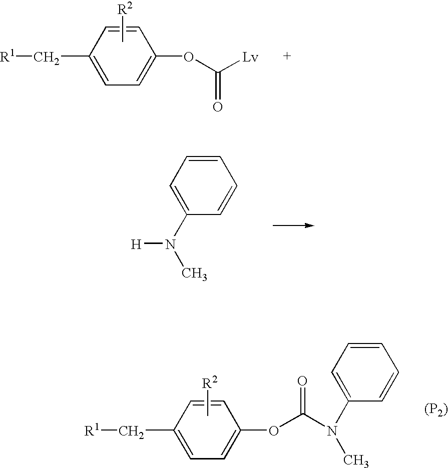
and isolating the disubstituted carbamate.

57. The process according to claim 56, wherein Lv is Cl.

58. The process according to claim 56, wherein said solvent is selected from the group consisting of diethyl ether, tetrahydrofuran and dichloromethane.

59. The process according to claim 56, wherein said base is selected from the group consisting of trimethylamine, triethylamine, ethyl-diisopropyl-amine and 1,4-diazabicyclo[2.2.2]octane.

60. The process according to claim 56, wherein said base is present as a functionality in one or both of the substituents R⁶ and R⁷, thus forming a salt with the acid H-Lv.
